SEQUENCE LISTING (18 sequences)

SEQ ID NO: 1 - protein, 12 aa, Artificial Sequence
  Description of Artificial Sequence Synthetic peptide
MEWSLEKGYTIK

SEQ ID NO: 2 - protein, 12 aa, Artificial Sequence
  Description of Artificial Sequence Synthetic peptide
WGWSLSHGYQVK

SEQ ID NO: 3 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 4 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 5 - protein, 374 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 6 - protein, 189 aa, Cowpea mosaic virus
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 7 - protein, 188 aa, Physalis mottle virus
MDSSEVVKVKQASIPAPGSILSQPNTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 8 - protein, 5 aa, Artificial Sequence
  Description of Artificial Sequence Synthetic peptide
GGGSC

SEQ ID NO: 9 - protein, 268 aa, Sesbania mosaic virus
MAKRLSKQQLAKAIANTLETPPQPKAGRRRNRRRQRSAVQQLQPTQAGISMAPSAQGAMVRIRNPAVSSSRGGITVLTHSELSAEIGVTDSIVVSSELVMPYTVGTWLRGVAANWSKYSWLSVRYTYIPSCPSSTAGSIHMGFQYDMADTVPVSVNQLSNLRGYVSGQVWSGSAGLCFINGTRCSDTSTAISTTLDVSKLGKKWYPYKTSADYATAVGVDVNIATPLVPARLVIALLDGSSSTAVAAGRIYCTYTIQMIEPTASALNN

SEQ ID NO: 10 - protein, 11 aa, Artificial Sequence
  Description of Artificial Sequence Synthetic peptide
ASSHIHHGGGS

SEQ ID NO: 11 - protein, 11 aa, Artificial Sequence
  Description of Artificial Sequence Synthetic peptide
HNHMADPGGGS

SEQ ID NO: 12 - protein, 14 aa, Artificial Sequence
  Description of Artificial Sequence Synthetic peptide
ACNSHRHGACGGGS

SEQ ID NO: 13 - protein, 14 aa, Artificial Sequence
  Description of Artificial Sequence Synthetic peptide
ACNSVHQHHCGGGS

SEQ ID NO: 14 - protein, 14 aa, Artificial Sequence
  Description of Artificial Sequence Synthetic peptide
ACPTGLHHACGGGS

SEQ ID NO: 15 - protein, 14 aa, Artificial Sequence
  Description of Artificial Sequence Synthetic peptide
ACTPKNNHSCGGGS

SEQ ID NO: 16 - protein, 11 aa, Artificial Sequence
  Description of Artificial Sequence Synthetic peptide
MDPHAHNGGGS

SEQ ID NO: 17 - protein, 17 aa, Artificial Sequence
  Description of Artificial Sequence Synthetic peptide
MEWSLEKGYTIKGGGSC

SEQ ID NO: 18 - protein, 17 aa, Artificial Sequence
  Description of Artificial Sequence Synthetic peptide
WGWSLSHGYQVKGGGSC

(19) United States
(12) Patent Application Publication
(10) Pub. No.: US 2024/0209325 A1
(43) Pub. Date: Jun. 27, 2024

(54) CANCER PROPHYLAXIS AND THERAPY USING TARGETED VIRAL NANOPARTICLES

(71) Applicant: The Regents of the University of California, Oakland, CA (US)

(72) Inventors: Nicole Steinmetz, La Jolla, CA (US); Jooneon Park, La Jolla, CA (US); Young Hun Chung, La Jolla, CA (US)

(21) Appl. No.: 18/554,979

(22) PCT Filed: Apr. 15, 2022
(86) PCT No.: PCT/US22/25071

§ 371 Date: Oct. 11, 2023

Related U.S. Application Data
(60) Provisional application No. 63/176,012, filed on Apr. 16, 2021.

(51) Int. Cl. C12N 7/00; A61K 39/12; A61P 35/00; A61K 9/51; A61K 39/00

(52) U.S. Cl. CPC C12N 7/00; A61K 39/12; A61P 35/00; A61K 9/51; A61K 2039/5258; C12N 2770/14023; C12N 2770/14034; C12N 2770/18023; C12N 2770/18034

(57) ABSTRACT

Methods and compositions for the treatment of cancer provided herein. The compositions and methods contain a nanoparticle and a peptide that targets S100A9.

### CROSS-REFERENCE TO RELATED APPLICATION

This application claims priority under 35 U.S.C. § 119(e) to U.S. Ser. No. 63/176,012, filed Apr. 16, 2021, the contents of which are incorporated herein by reference.

### STATEMENT OF GOVERNMENT SUPPORT

This invention was made with government support under CA218292, HL137674, and CA224605 awarded by the National Institutes of Health. The government has certain rights in the invention.

### BACKGROUND

The following discussion of the background is merely provided to aid the reader in the understanding the disclosure and is not admitted to describe or constitute prior art to the present disclosure. Throughout and within this disclosure, various patent and technical publications are referenced by an identifying citation or an Arabic number, the full bibliographic citation for which can be found immediately preceding the claims. These disclosures are incorporated herein to more fully describe the state of the art to which this disclosure pertains.

Metastatic cancer remains a challenge to treat and diagnose regardless of the cancer's origin. For instance, the median survival time of breast cancer patients with metastatic recurrences is 2-3 years.¹ Metastatic melanoma tumors are similar in their aggressiveness and prognosis of the disease becomes very difficult once metastasis has been achieved by the primary tumor.² Metastasis to the lungs remains one of the most common forms of metastasis in both breast cancer and melanoma. In autopsy studies, lung metastasis in breast cancer was found in 57-77% of patients and found between 10-40% in melanoma patients.^{3,4} Once lung metastasis occurs, the median survival rate of breast cancer patients is 22 months while in melanoma, overall survival is around 13 months.^{4,5} Systemic chemotherapeutics are the primary treatment for metastatic disease but are limited by dose-limiting toxicity, the development of drug resistance, adverse effects and suboptimal dosing can lead to drug resistance.⁶ Further, chemotherapy induces long-term side effects—in melanoma, chemotherapy can lead to skin and gastrointestinal toxicity.⁷ Prognosis and treatment of metastatic cancer continues to be one of the most difficult and challenging areas of oncology. This disclosure satisfies this need and provides related advantages as well.

### SUMMARY OF THE DISCLOSURE

This disclosure provides a nanoparticle comprising, or alternatively consisting essentially of a virus or virus like particle (VLP) and a peptide that recognizes and binds S100A9. In one aspect, the virus or VLP is from a plant virus from the group of the genus Bromovirus, Comovirus, or Tymovirus. In another aspect, the plant virus is selected from Cowpea chlorotic mottle virus (CCMV), Cowpea mosaic virus (CPMV), or Physalis mottle virus (PhMV).

In a further aspect, the virus or VLP has an exposed lysine side chain. In another aspect, the peptide comprises, or alternatively consists essentially of, or yet further consists of a c-terminal cysteine that can be introduced via a linker. An example of such a linker includes the peptide GGGSC. In one aspect, the lysine side chain is conjugated to an N-hydroxysuccinimide (NHS) ester and the maleimide of a maleimide-polyethylene glycol₈ is conjugated with the c-terminal cysteine of the peptide. In one embodiment, the peptide that recognizes and binds S100A9 comprises or consists of a peptide having the amino acid of one or more of SEQ ID NO: 1 and/or 2, or 1, 2 or 10 to 15 or a immunogenic fragment thereof, or an equivalent of each thereof that is at least 70% identical, or at least 80%, or at least 90% or at least 95%, or at least 98% or similar to one or more of SEQ ID NO: 1 and/or 2, or 1, 2 or 10 to 15 or a immunogenic fragment thereof, respectively, or the immunogenic fragment thereof. In a further aspect, SEQ ID NO: 1 and/or 2, equivalent or fragment thereof further comprises, consists essentially of, or consist of a linker with a c-terminal cysteine, e.g., GGGSC.

Also provided herein are isolated polynucleotides encoding the virus, VLP, nanoparticle of this disclosure or an equivalent thereof. In one aspect, as provided herein, is a vector, comprising, or alternatively consisting essentially of, or yet further consisting of the virus, VLP, polynucleotide of this disclosure, optionally linked to regulatory or other elements for the expression of the virus, VLP, nanoparticle. For the production of vectors, the vector genome is expressed from a DNA construct encoding it in a host cell. Thus in another aspect, as provided herein, is a host cell, comprising, or alternatively consisting essentially of, or yet further consisting of the polynucleotide of this disclosure.

Further provided herein, are a plurality of virus, VLP, or nanoparticles, where the virus, VLP, or nanoparticles are the same or different from each other.

In one aspect, provided herein is a composition comprising, or alternatively consisting essentially of, or yet further consisting of the virus, VLP, nanoparticle, polynucleotide, vector and/or host cell. In another aspect, provided herein is a composition comprising, or alternatively consisting essentially of, or yet further consisting of a carrier and one or more of the nanoparticle, polynucleotide, vector and/or the host cell of this disclosure.

In a further aspect, provided herein is a method for inducing an immune response the method comprising, or alternatively consisting essentially of, or yet further consisting of administering to the subject one or more of the virus, VLP, nanoparticle, polynucleotide, vector, the composition and/or the host cell of this disclosure. Also provided herein is a method for targeting the tumor microenvironment to reverse immunosuppression of a tumor that optionally secretes S100A, the method comprising, or consisting essentially of, or consisting of contacting the tumor microenvironment with the virus, VLP, nanoparticle, polynucleotide, vector, the composition and/or the host cell of this disclosure. The contacting can be in vitro or in vivo. The S100A9 target can be secreted into the tumor microenvironment, but in one aspect, not necessarily expressed on the surface of tumor cells. Targeting the tumor microenvironment will interact and contact immune cells to reverse immunosuppression and launch anti-tumor immunity through activation of the innate and then adaptive immune system.

Methods to determine when tumor has been inhibited are known in the art. In vitro, the method can be used to screen for combination therapies or for personalized treatments for animals, mammals and human. In one aspect, the cancer cell is any of the cancer cells described herein, including primary and metastatic cells. In vivo, the method comprises administering an effective amount of the composition as described herein and can be used to test for new drug combinations (in an animal model) or for therapy itself. The treatment can be combined with other therapies and therefore can be a first line, second line, third line, fourth line or fifth line therapy. Methods to determine effectiveness of the therapy include reduction in tumor size or burden, prolonged progression-free survival, prolonged overall survival and reduced toxicity. These methods can be combined with other clinical markers, e.g., a reduction in a tumor marker, e.g., CA125.

One embodiment of the disclosure relates to a method for treating or preventing cancer or metastasis in a subject in need thereof, comprising, or alternatively consisting essentially of, or yet further consisting of administering to the subject in need thereof one or more of the virus, VLP, nanoparticle, polynucleotide, vector, the composition and/or the host cell of this disclosure. In one aspect, the cancer is any of the cancer cells described herein, including primary and metastatic cancer. When practiced in a non-human subject or a human subject, the method comprises administering an effective amount of the composition as described herein. In an animal model the method can be used to test for new drug combinations or for therapy itself. The treatment can be combined with other therapies and therefore can be a first line, second line, third line, fourth line or fifth line therapy. Methods to determine effectiveness of the therapy include reduction in tumor size or burden, prolonged progression-free survival, prolonged overall survival and reduced toxicity. These methods can be combined with other clinical markers, e.g., a reduction in a tumor marker, e.g., CA125.

In a further aspect, provided herein is a method for altering an immune cell profile in lungs of a subject, the method comprising, or alternatively consisting essentially of, or yet further consisting of the virus, VLP, nanoparticle, polynucleotide, vector, the composition and/or the host cell of this disclosure. When practiced in a non-human subject or a human subject, the method comprises administering an effective amount of the composition as described herein. In an animal model the method can be used to test for new drug combinations or for therapy itself. The treatment can be combined with other therapies and therefore can be a first line, second line, third line, fourth line or fifth line therapy. Methods to determine effectiveness of the therapy are known in the art and described herein.

In one particular aspect, the present disclosure provides kits for performing the methods of this disclosure as well as instructions for carrying out the methods of the present disclosure. The kit comprises, or alternatively consists essentially of, or yet further consists of one or more of the virus, VLP, nanoparticle, polynucleotide, vector, the composition and/or the host cell of this disclosure In a further aspect, the instruction for use provide directions to conduct any of the methods disclosed herein.

### BRIEF DESCRIPTION OF THE DRAWINGS

CPMV bioconjugation strategy. CPMV is first extracted from infected black-eyed pea No. 5 plants. The large and small coat proteins are shown in light and dark grey; surface exposed Lys side chains are highlighted as black spheres. The H6/G3 peptides with C-terminal Cys side chain (the linker is underlined) were then conjugated to CPMV using an SMPEG₈ linker via NHS-maleimide chemistry. CPMV images and chemical structures were drawn with UCSF Chimera and ChemDraw software. The image of the leaf was adapted from Biorender.com.

FIGS. 2A-2E: Characterization of CPMV, peptide-conjugated CPMV, and fluorescent CPMV particles. (FIG. 2A) SDSPAGE of the CPMV particles. The arrows point to H6/G3 peptide-modified coat proteins. One arrow points to the large coat protein (42 kDa) and the corresponding peptide-conjugated coat protein, and the other arrow points to the small coat protein (24 kDa) and the corresponding peptide-conjugated coat protein. (FIG. 2B) DLS measurements of the CPMV particles. The box in black is displaying the average diameter in nm of the particles (FIG. 2D) and the polydispersity index (PDI). (FIG. 2C) TEM images of uranyl acetate-stained CPMV particles. Scale bars represent 100 nm. (FIG. 2D) UV-VIS of the fluorescent Cy5-conjugated CPMV particles. The boxed insets are displaying the number of conjugated Cy5 particles per CPMV particle. (FIG. 2E) FPLC measurements of the fluorescent and peptide-conjugated CPMV particles. The inset is indicating the 260/280 nm ratio at the peak of the FPLC curve. Corresponding CCMV data are shown in FIG. 8E).

FIGS. 3A-3E: Biodistribution and localization of fluorescent CPMV and CCMV nanoparticles following administration. (FIG. 3A) Schematic and timeline of the biodistribution study. (FIG. 3B) IVIS imaging of lungs following CPMV and CCMV nanoparticle injection. FIG. 3B was presented with a color gradient from deep red (10 k) to yellow (30K). To help clarify the grey scale version of FIG. 3B: Lung results have true color for CPMV-G3 for middle and bottom and CPMV-H6 in all three positions (FIG. 3C, FIG. 3D) Quantitative analysis of the fluorescence signal from the organs after CPMV (FIG. 3C) and CCMV (FIG. 3D) nanoparticle injection. (FIG. 3E) Confocal imaging indicates colocalization of the CPMV-Cy5-G3 particles with S100A9. Scale bar represents 25 μm. DAPI staining is shown in blue while S100A9 and CPMV particles are shown in teal and yellow, respectively. The bottom right image is the merged image showing co-localization between the S100A9 and the CPMV-Cy5-G3. *=p<0.05, **=p<0.01, ***=p<0.001, ****=p<0.0001, ns=not significant. All analyses were done by one-way ANOVA.

FIGS. 4A-4I: CPMV particles show immunoprophylaxis in C57BL/6J mice challenged i.v. with B16F10 melanoma or 4T1-Luc TNBC cells. (FIG. 4A) Schematic and timeline of the B16F10 prophylaxis study. (FIG. 4B) Harvested lungs were fixed and imaged before manual tumor counting. (FIG. 4C) Quantitative analysis of the number of tumor nodules found on the surface of the lungs. The middle line indicates the mean number of tumor nodules (FIG. 4D) Repeated B16F10 prophylactic immunotherapy study including an H6 peptide only control. The middle line indicates the mean number of tumor nodules. (FIG. 4E) H&E images of the harvested lungs. The dark purple spots are indicative of the B16F10 tumor nodules in the lungs. (FIG. 4F) Quantitative analysis of the H&E pictures in (FIG. 4E). The ratio of tumor cells to total cells within the H&E images were plotted. The images were analyzed using QuPath software. (FIG. 4G) Schematic and timeline of the 4T1-Luc prophylaxis study. (FIG. 4H) Harvested lungs were fixed in Bouin's solution before manual tumor counting. The tumor nodules are highlighted by the red arrows. (FIG. 4I) Quantitative analysis of the tumor nodules from the lungs in h). *=p<0.05, **=p<0.01, ***=p<0.001, ****=p<0.0001, ns=not significant. All analyses were done by one-way ANOVA.

FIGS. 5A-5E: S100A9-targeted CPMV immunotherapy against lung metastasis from i.v. injected B16F10 melanoma and 4T1-Luc breast cancer cells in mice. (FIG. 5A) Treatment schedule of the metastatic B16F10 melanoma model using C57BL/6J mice and therapeutic administration of CPMV and CPMV-H6. (FIG. 5B) Quantitative analysis of tumor nodules counted in lungs harvested post-treatment. (FIG. 5C) Treatment schedule of the metastatic 4T1-Luc breast cancer model using Balb/c mice. (FIG. 5D) Quantitative luminescence of the tumors following ROI measurements of the images from (FIG. 5E) Luminescent imaging of the 4T1-luc tumors taken on the IVIS. The mice were imaged every two days following 150 mg/kg i.p. injection of D-luciferin, and the luminescence was calculated using ROI measurements from the Living Image 3.0 software. One representative image taken on the IVIS on day 9 is shown. *=p<0.05, **=p<0.01, ***=p<0.001, ****=p<0.0001, ns=not significant. All analyses were done by either one or two-way ANOVA.

Original data for FIG. 5E involves a color gradient from blue (<10K) up to red (30K). For clarity, the PBS data has red centers in the left most and right most panels. The CPMV data has a dark center only in the right most panel. All other data is in gray scale.

FIGS. 6A-6C: Immunogenicity assays of CPMV and CCMV particles. (FIG. 6A) A RAW-BLUE™ assay comparing the immunogenicity between wild type CPMV, peptide-conjugated CPMV, and the peptide only controls. The CPMV and the peptide-conjugated CPMV particles were strong TLR and/or NOD agonists while the peptides by themselves were not indicating that the peptides themselves do not impart any significant immunotherapeutic effects. (FIG. 6B) RAWBLUE™ assay comparing the immunogenicity of CPMV to CCMV. The CPMV was much more immunogenic compared to CCMV, which helps to explain why in the previous studies, CPMV was able to significantly reduce tumor burden while CCMV did not. (FIG. 6C) FACS analysis of the immune cell profile following CPMV injection. C57BL/6J mice were i.v. injected with CPMV, CPMV-H6, CPMV-G3, and PBS and the lungs were harvested and analyzed. CPMV-H6 showed greater DC recruitment and DC activation while CPMV-G3 did not recruit more DCs, but did activate them significantly more than controls. CPMV-H6 further recruited neutrophils and macrophages. Phenotypic assessment found that CPMV-H6 and CPMV-G3 both showed significant activation of M1 anti-tumor macrophages. *=p<0.05, **=p<0.01, ***=p<0.001, *=p<0.0001, ns=not significant. All analyses were done by one-way ANOVA.

FIG. 7: CCMV bioconjugation strategy. CCMV is first extracted from infected black-eyed pea No. 5 plants. Surface exposed lysines are highlighted as black spheres. The H6/G3 peptides with C-terminal Cys side chain (the linker is underlined) were then conjugated to CCMV using an SMPEG₈ linker via NHS-maleimide chemistry. CCMV images and chemical structures were drawn with UCSF Chimera and ChemDraw software. The image of the leaf was adapted from Biorender.com.

FIGS. 8A-8E: Characterization of CCMV, peptide-conjugated CCMV, and fluorescent CCMV particles. (FIG. 8A) SDS-PAGE of the CCMV particles. The purple arrow points to H6/G3 peptide-modified coat proteins. The blue arrow points to the CCMV coat protein (21 kDa), and the red arrow points to the H6/G3 peptides. (FIG. 8B) DLS measurements of the CCMV particles. The box in black is displaying the average diameter in nm of the particles (D) and the polydispersity index (PDI). The low PDI indicates minimal aggregation of the CCMV particles following bioconjugation. (FIG. 8C) TEM images of uranyl acetate-stained CCMV particles. Scale bars represent 100 nm. (FIG. 8D) UV-VIS of the fluorescent Cy5-conjugated CCMV particles. The boxed insets are displaying the number of conjugated Cy5 particles per CCMV particle. (FIG. 8E) FPLC measurements of the dual fluorescent and peptide-conjugated CCMV particles. The inset is indicating the 260/280 nm ratio at the peak of the FPLC curve.

FIGS. 9A-9D: Additional characterization of the non-fluorescent CPMV and CCMV particles. (FIG. 9A, FIG. 9B) 1.2% (w/v) agarose gel of the CPMV (FIG. 9A) and CCMV (FIG. 9B) particles. Data indicate that particles remain intact and do not aggregate. FPLC graph of the CPMV (FIG. 9C) and CCMV (FIG. 9D) particles using a Superose 6 column and ÄTKA purifier. The inset is indicating the absorbance 260/280 nm ratio at the peak of the FPLC curve; an A260/280 ratio of 1.8 is indicative of intact particle preparations. The elution profiles are consistent with intact particles, although the left-shifted curve for CPMV may indicate some level of aggregation.

FIGS. 10A-10B: Additional characterization of dual-tagged peptide and fluorescent CPMV particles. (FIG. 10A) SDS/PAGE gels of fluorescent CPMV particles. The Coomassie staining indicates successful conjugation to the peptides (as seen by the higher molecular weight bands) and the fluorescence (imaged at wavelength 647 nm) indicates that both the small and large coat proteins were successfully conjugated to the Cy5 fluorophore. (FIG. 10B) 1.2% (w/v) agarose gels of the native and dual-tagged CPMV particles show that the particles remain intact. There is also minimal aggregation and no broken particles as indicated by the single bands.

FIGS. 11A-11C: Biodistribution of fluorescent CPMV and CCMV nanoparticles following administration. (FIG. 11A) Schematic and timeline of the biodistribution study. (FIG. 11B, FIG. 11C) IVIS imaging of kidneys, spleens, and livers following CPMV (FIG. 11B) and CCMV (FIG. 11C) nanoparticle injection.

FIGS. 12A-12B: 4T1-Luc immunoprophylaxis study. (FIG. 12A) Schematic and treatment schedule of CPMV as an immunoprophylaxis in 4T1-Luc metastatic breast cancer. (FIG. 12B) Luminescent imaging of the 4T1-Luc tumors taken every 3 days. D-luciferin was injected i.p. at a concentration of 150 mg/kg to enable luminescent imaging. Additionally, the mice were weighed and checked for any signs of noticeable weight loss. PBS mice were sacrificed by day 21 after meeting clinical endpoints and could not be imaged. *=p<0.05, **=p<0.01, ***=p<0.001, ****=p<0.0001, ns=not significant. All analyses were done by one-way ANOVA. Original data for FIG. 12B involves color gradients for blue (4K or 5K) to red (8K or 25K). The third mouse from the left for PBS Day 15 and the first and third mouse from the left for PBS Day 18 contain red data points. The third mouse from the left for Day 15 CPMV-G3 contains red data points.

FIGS. 13A-13C: S100A9-targeted CPMV immunotherapy against lung metastasis from i.v. injected 4T1-Luc breast cancer cells in mice. (FIG. 13A) Schematic and treatment schedule of the 4T1-Luc breast cancer model using Balb/c mice. (FIG. 13B) Kaplan-Meier curve of the mice from the 4T1-Luc immunotherapy study. The mice were sacrificed when they reached their clinical endpoints. Wild type CPMV increased the median time of survival by one day while the CPMV-H6 treatment increased the median time of survival by 3 days. (FIG. 13C) Luminescent imaging of the 4T1-Luc tumors taken on the IVIS. The mice were imaged every two days following 150 mg/kg i.p. injection of D-luciferin, and the luminescence was calculated using ROI measurements from the Living Image 3.0 software. *=p<0.05, **=p<0.01, ***=p<0.001, ****=p<0.0001, ns=not significant. All analyses were done by one-way ANOVA.

FIG. 14: The gating strategy used in (FIG. 6C) of the flow cytometry analysis of CPMV particles' ability to stimulate innate immune cells.

FIGS. 15A-15C: Liver enzyme assays to determine liver toxicity of the CPMV i.v. injections. (FIG. 15A) Schematic of the ALT and AST assays. (FIG. 15B) ALT Assay (FIG. 15C) AST assay. The dotted lines in the ALT and AST assays represent the normal levels of ALT and AST in the blood of mice. Due to the strong accumulation of the CPMV particles in the liver (FIG. 3B, FIG. 3C), Applicant tested whether or not the CPMV particles were hepatotoxic in the mice. C57BL/6 mice (n=5-7) were injected with 200 μg of CPMV, CPMV-H6, and CPMV-G3 as well as PBS, H6, and G3 controls (FIG. 15). After 1, 3, and 7 days, the blood of the mice were collected and the sera was collected with centrifugation. The sera were then tested for both ALT and AST activity, which are both enzymes released in the blood following injection of hepatotoxic molecules.⁶⁹ The assays show that for both the ALT and AST assays, injection of CPMV-H6 and CPMV-G3 led to small increases in the ALT and AST systemic concentrations after 1 day (FIG. 15B, FIG. 15C). However, by day 3 the enzyme levels were within the physiological ranges and the same was true for day 7. After significant liver damage, usually ALT and AST concentrations spike to 50 and 10-20× the normal levels¹—our CPMV injections led to levels well below that range.

FIGS. 16A-16E: Characterization of CPMV, CPMV-H6, and CPMV-G3. (FIG. 16A) UV-VIS (FIG. 16B) SDS-PAGE (FIG. 16C) Agarose (FIG. 16D) DLS (FIG. 16E) FPLC.

FIGS. 17A-17B: Detection of S100A8/9 within the IP space. (FIG. 17A) Injection schedule. (FIG. 17B) ELISA data from IP gavage indicating that S100A8/9 levels within the IP space increase significantly following CT26-Luc (colon cancer) injection.

FIGS. 18A-18D: CT26-Luc treatment with S100A9-targeted CPMV particles. Treatment with CPMV-H6/G3 significantly extends survival and slows the growth of the colon cancer. (FIG. 18A) Injection schedule. (FIG. 18B) Body weight fold change of mice (FIG. 18C) Circumference fold change of mice. (FIG. 18D) Survival curve of mice.

FIGS. 19A-19C: Targeting of IP tumors using CPMV-H6/G3. (FIG. 19A) Injection Schedule. (FIG. 19B) Quantitative fluorescent measurements of the IP tumors from IVIS imaging. Data indicates a trend for both the CPMV-H6 and CPMV-G3 in targeting the tumors. (FIG. 19C) Ex vivo imaging of harvested tumors following CT26-Luc IP injection.

FIGS. 20A-20D: Additional targeting data. (FIG. 20A) Lungs, (FIG. 20B) Kidneys, (FIG. 20C) Spleen, (FIG. 20D) Liver.

### DETAILED DESCRIPTION

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+ T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated. In some embodiments, the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, “cancer” or “malignancy” or “tumor” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

As used herein, a “metastatic cancer” is a cancer that spreads from where it originated to another part of the body.

As used herein, a “cancer cell” are cells that have uncontrolled cell division and form solid tumors or enter the blood stream.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

An agent of the present disclosure can be administered for therapy by any suitable route of administration. It will also be appreciated that the optimal route will vary with the condition and age of the recipient, and the disease being treated.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-1 2, RPMI 1640, and Iscove's Modified Dulbecco's Medium (IMDM).

S100 calcium-binding protein A9 (S100A9; also known as migration inhibitory factor-related protein 14 or MRP14 or calgranulin B) is a protein involved in cellular processes such as cell cycle progression and differentiation and a central mediator of inflammation in cancer and other diseases.⁸,⁹ It is a calcium-binding protein that regulates inflammation and while there is some level of endogenous S100A9 expression in the squamous epithelium and mucosal tissues,⁹,¹⁰ it becomes overexpressed in many different forms of cancer including breast, ovarian, skin, bladder, pancreatic, gastric, esophageal, colon, glioma, cervical, hepatocellular, and thyroid.^{8,11_13} It is most commonly found in its heterodimer form with S100A8, but can also be found as a homodimer.^{12,14,15} S100A8/9 complexes are also found in mice and extensive biochemical characterization has demonstrated functional equivalency with its human counterpart.¹⁶ S100A9 expression is heavily linked with tumor aggressiveness and tumorigenesis through the activation of the nuclear factor-κB (NF-κB) and mitogen-activated protein kinase (MAPK) pathways, which are responsible for inflammation-induced cancer development and uncontrolled cell proliferation respectively.¹⁷,¹⁸ It is mainly expressed and secreted by MDSCs, which promotes further accumulation of MDSCs via autocrine pathways into the tumor microenvironment (TME) in an expanding and cyclic fashion.¹⁸ MDSCs suppress the immune response within the TME through reprogramming of the TME into a protumor phenotype, and tumors soon begin establishing S100A9 gradients of myeloid cell migration.^{19,20} All the downstream effects of S100A9 establishment within the TME point to a clear link between S100A9 expression and tumor progression and metastasis.^{8,11_13} Antibodies targeting S100A9 are being investigated as novel targeted therapeutics.^{21,22} Kwak et al. generated peptides called H6 (MEWSLEKGYTIK SEQ ID NO:1) and G3 (WGWSLSHGYQVK SEQ ID NO: 2) that were found through phage display and targeted S100A9.²² Kwak et al. fused these peptides to the Fc region of mouse IgG2b antibodies (termed peptibodies) and found that these peptibodies were successful in depleting MDSCs in multiple tumor models in the blood, spleen, and tumor leading to tumor growth inhibition.²² Similarly, neutralizing antibodies blocking S100A9 inhibited MDSC accumulation and decreased expression of serum amyloid 3, a recruiter of circulating tumor cells.²¹ Outside of these therapies, targeting S100A9 in cancer immunotherapy is a novel concept that has not been explored to the best of Applicant's knowledge.^{8,22}

An exemplary S100A9 sequence (reproduced from NP_002956.1, (www.ncbi.nlm.nih.gov/protein/NP_002956.1 (accessed on Mar. 10, 2022)) is:

(SEQ ID NO: 3)
MTCKMSQLER NIETIINTFH QYSVKLGHPD TLNQGEFKEL
VRKDLQNFLK KENKNEKVIE HIMEDLDTNA DKQLSFEEFI
MLMARLTWAS HEKMHEGDEG PGHHHKPGLG EGTP

Other peptides that target or recognize and bind S100A9, include without limitation the peptides identified below as MTP2 through MTP10. Peptide construction and screening is described in Park et al. (2021) Biomacromolecules 22:2582-2594, incorporated herein by reference.

| label | sequence | ID No. | KD (μM) |
|---|---|---|---|
| scrambled | MDPHAHNGGGS | 16 | no binding |
| MTP2 | ASSHIHHGGGS | 10 | 77.6 |
| MTP3 | HNHMADPGGGS | 11 | 116.8 |
| MTP5 | ACNSHRHGACGGGS | 12 | 67.6 |
| MTP6 | ACNSVHQHHCGGGS | 13 | 39.6 |
| MTP9 | ACPTGLHHACGGGS | 14 | 190.9 |
| MTP10 | ACTPKNNHSCGGGS | 15 | 191.0 |

### Virus and Virus-Like Particles (VLPs)

VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs can also be engineered, e.g., comprising, or consisting essentially of, or yet further consisting of, one or more viral proteins that comprise, or consists essentially of, or yet further consists of, a modification. Methods for producing VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354: 53073, 2012).

In some embodiments, the virus or VLP is derived from Cowpea chlorotic mottle virus (CCMV). CCMV is a spherical plant virus that belongs to the Bromovirus genus. Several strains have been identified and include, but not limited to, Car1 (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean (S) (Kuhn, 1968. Phytopathology 58:1441-1442), mild (M) (Kuhn, 1979. Phytopathology 69:621-624), Arkansas (A) (Fulton, et al., 1975. Phytopathology 65: 741-742), bean yellow stipple (BYS) (Fulton, et al., 1975. Phytopathology 65: 741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72(9): 768-770).

In some instances, the virus or VLP from CCMV comprise, or consists essentially of, or yet further consists of, a plurality of capsid proteins. In some instances, the capsid protein is a wild-type CCMV capsid, optionally expressed by Car1, Car2, type T, soybean (S), mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprise, or consists essentially of, or yet further consists of, the sequence as set forth in the UniProtKB ID P03601:

(SEQ ID NO: 4)
MSTVGTGKLTRAQRRAAARKNKRNTRVVQPVIVEPIASGQGKAIKAWTGY
SVSKWTASCAAAEAKVTSAITISLPNELSSERNKQLKVGRVLLWLGLLPS
VSGTVKSCVTETQTTAAASFQVALAVADNSKDVVAAMYPEAFKGITLEQL
TADLTIYLYSSAALTEGDVIVHLEVEHVRPTFDDSFTPVY,

or an equivalent thereof.

In some cases, the virus or VLP from CCMV is prepared by the method as described in Ali et al., “Rapid and efficient purification of Cowpea chlorotic mottle virus by sucrose cushion ultracentrifugation,” Journal of Virological Methods 141: 84-86 (2007).

In some embodiments, the virus or VLP is derived from Cowpea mosaic virus (CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the virus or VLP from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

(SEQ ID NO: 5)
MEQNLFALSLDDTSSVRGSLLDTKFAQTRVLLSKAMAGGDVLLDEYLYDV
VNGQDFRATVAFLRTHVITGKIKVTATTNISDNSGCCLMLAINSGVRGKY
STDVYTICSQDSMTWNPGCKKNFSFTFNPNPCGDSWSAEMISRSRVRMTV
ICVSGWTLSPTTDVIAKLDWSIVNEKCEPTIYHLADCQNWLPLNRWMGKL
TFPQGVTSEVRRMPLSIGGGAGATQAFLANMPNSWISMWRYFRGELHFEV
TKMSSPYIKATVTFLIAFGNLSDAFGFYESFPHRIVQFAEVEEKCTLVFS
QQEFVTAWSTQVNPRTTLEADGCPYLYAIIHDSTTGTISGDFNLGVKLVG
IKDFCGIGSNPGIDGSRLLGAIAQ,

or an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprise, or consists essentially of, or yet further consists of, the sequence as set forth in the UniProtKB ID P03599 (residues 834-1022):

(SEQ ID NO: 6)
GPVCAEASDVYSPCMIASTPPAPFSDVTAVTFDLINGKITPVGDDNWNTH
IYNPPIMNVLRTAAWKSGTIHVQLNVRGAGVKRADWDGQVFVYLRQSMNP
ESYDARTFVISQPGSAMLNFSFDIIGPNSGFEFAESPWANQTTWYLECVA
TNPRQIQQFEVNMRFDPNFRVAGNILMPPFPLSTETPPL,

or an equivalent thereof.

In some embodiments, the virus or VLP is derived from Physalis mottle virus (PhMV). PhMV is a single stranded RNA virus that belongs to the genus Tymovirus. In some instances, the virus or VLP from PhMV comprises, or consists essentially of, or yet further consists of, a plurality of coat proteins. In some instances, the coat protein is a wild-type PhMV coat protein. In other instances, the coat protein is a modified coat protein, e.g., comprising, or consisting essentially of, or yet further consisting of, one or more substitutions, insertions, and/or deletions. In some cases, the PhMV coat comprise, or consists essentially of, or yet further consists of, the sequence as set forth in the UniProtKB ID P36351:

(SEQ ID NO: 7)
MDSSEVVKVKQASIPAPGSILSQPNTEQSPAIVLPFQFEATTFGTAETAA
QVSLQTADPITKLTAPYRHAQIVECKAILTPTDLAVSNPLTVYLAWVPAN
SPATPTQILRVYGGQSFVLGGAISAAKTIEVPLNLDSVNRMLKDSVTYTD
TPKLLAYSRAPTNPSKIPTASIQISGRIRLSKPMLIAN,

or an equivalent thereof.

In some embodiments, the virus or VLP is derived from Sesbania mosaic virus (SeMV). SeMV is a positive stranded RNA virus that belongs to the genus Sobemovirus. In some instances, the virus or VLP from SeMV comprise, or consists essentially of, or yet further consists of, a plurality of capsid proteins. In some instances, the capsid protein is a wild-type SeMV capsid protein. In other instances, the capsid protein is a modified capsid protein, e.g., comprising, or consisting essentially of, or yet further consisting of, one or more substitutions, insertions, and/or deletions. In some cases, the SeMV capsid comprise, or consists essentially of, or yet further consists of, the sequence as set forth in the UniProtKB ID Q9EB06:

(SEQ ID NO: 9)
MAKRLSKQQLAKAIANTLETPPQPKAGRRRNRRRQRSAVQQLQPTQAGIS
MAPSAQGAMVRIRNPAVSSSRGGITVLTHSELSAEIGVTDSIVVSSELVM
PYTVGTWLRGVAANWSKYSWLSVRYTYIPSCPSSTAGSIHMGFQYDMADT
VPVSVNQLSNLRGYVSGQVWSGSAGLCFINGTRCSDTSTAISTTLDVSKL
GKKWYPYKTSADYATAVGVDVNIATPLVPARLVIALLDGSSSTAVAAGRI
YCTYTIQMIEPTASALNN,

or an equivalent thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a virus or VLP.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid. \Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some instances, a virus or VLP described herein further comprise, or consists essentially of, or yet further consists of, a label or a tag, e.g., such as a detectable label. A detectable label can be attached to, e.g., to the surface of a virus or VLP.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe (III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the virus or VLP or nanoparticle. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

Also provided herein is the virus, VLP, or nanoparticle as described herein further comprising, or consisting essentially of, or yet further consisting of the peptide that recognizes and binds S100A9 and an additional therapeutic agent. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the virus, VLP, or nanoparticle with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the additional therapeutic agent comprise, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include:
PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736;
PD-L2 inhibitors such as GlaxoSmithKline's AMP-224 (Amplimmune), and rHIgM12B7;
PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd;
CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam;
LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;
B7-H3 inhibitors such as MGA271;
KIR inhibitors such as Lirilumab (IPH2101);
CD137 inhibitors such as urelumab (BMS-663513, Bristol-Myers Squibb), PF-05082566 (anti-4-1BB, PF-2566, Pfizer), or XmAb-5592 (Xencor);
PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, TLR1, TLR2, TLR3, TLR4, TLR5, TLR6, TLR7, TLR8, or TLR9. In some cases, the TLR ligand comprise, or consists essentially of, or yet further consists of, a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the virus or VLP or nanoparticle formulation described herein is administered in combination with a radiation therapy.

### Modes for Carrying out the Disclosure

### Nanoparticles

Applicant demonstrates herein that prophylaxis and treatment of lung metastasis could be achieved by targeting immunostimulatory nanoparticles to the lung, which specifically target S100A9 (otherwise known as myeloid-related protein 14). S100A9 is a calcium-binding protein implicated in tumor metastasis, progression, and aggressiveness that modulates the tumor microenvironment into an immunosuppressive state. S100A9 is expressed in and secreted by immune cells in the pre-metastatic niche as well as post-tumor development, therefore making it a suitable targeted for prophylaxis and therapy. S100A9-specific peptide ligands were selected and presented on immunostimulatory nanoparticles derived from cowpea mosaic virus (CPMV) and CCMV. Applicant also demonstrates that systemically administered, S100A9-targeted nanoparticles such as CPMV or CCMV homes to the lungs leading to recruitment of dendritic cells and neutrophils as well as polarizes macrophages into the M1 antitumor phenotype. Efficacy is demonstrated with prophylaxis and therapy in preventing and treating lung metastasis from melanoma and triple negative breast cancer (TNBC).

In some embodiments, a nanoparticle comprises, or alternatively consists essentially of, or yet further consists of a virus or VLP such as CPMV or CCMV and a S100A9 (otherwise known as myeloid-related protein 14) targeting peptide. In some embodiments, the S100A9 targeting peptide comprises, or alternatively consisting essentially of, or yet further consisting a peptide selected from H6 peptide or G3 peptide. In some embodiments, the virus, VLP, or nanoparticle comprises the amino acid of one or more of SEQ ID NO: 1 and/or 2, or 1, 2 or 10 to 15 an immunogenic fragment thereof, or an equivalent thereof at least 70% identical or similar to one or more of SEQ ID NO: 1 and/or 2, or 1, 2 or 10 to 15 or the immunogenic fragment thereof. In some aspect, the S100A9 targeting peptide further comprises a linker, that optionally contains a c-terminal cysteine, e.g., GGGSC. In some embodiments, the virus or VLP (e.g., CMPV or CCMV) has an exposed lysine side chain.

The nanoparticle, virus or VLP and/or targeting peptide can be detectably labeled for diagnostic or research purposes. Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ^{0.57}Co, ⁶⁰Cu, ⁶¹Cu, ⁶²Cu, ⁶⁴Cu, ⁶⁷Cu, ⁶⁷Ga, ⁶⁸Ga, ⁷²As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe (III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

Also provided herein is the virus or VLP as described herein further comprising, or consisting essentially of, or yet further consisting of the peptide that recognizes and binds S100A9 and an additional therapeutic agent. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone. The additional therapeutic can be conjugated to the virus or VLP using methods known in the art and as described herein.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol -Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-IBB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, TLR1, TLR2, TLR3, TLR4, TLR5, TLR6, TLR7, TLR8, or TLR9. In some cases, the TLR ligand comprise, or consists essentially of, or yet further consists of, a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some embodiments the peptide can be chemically conjugated or genetically fused to the CMPV or CCMV. Any bioconjugation or chemical conjugation method would be applicable for the conjugation. Non-limiting examples of chemical conjugation include conjugating a thiol-terminated peptide through a maleimide-PEG-NHS linker targeting lysine groups on the virus or VLP, e.g., CMPV or CCMV. In some embodiments, a lysine side chain is conjugated to a N-hydroxysuccinimide (NHS) ester and the maleimide of a maleimide-polyethylene glycol₈ is conjugated with the c-terminal cysteine of the targeting peptide. Azide/alkyne modified peptides and virus or VLP (CMPV or CCMV) and click chemistry can also be used for chemical conjugation. For bioconjugation such as genetic fusion, the peptide is added as N-terminal fusion in a CMPV or CCMV plasmid containing the entire VLP (e.g., CMPV or CCMV genome).

In some embodiments, the diameter of the nanoparticle disclosed herein, is from about 10 nm to 50 nm. In some embodiments, the diameter may range from about 10 nm, about 15 nm, about 20 nm, about 25 nm, about 30 nm, about 35 nm, about 40 nm, about 45 nm, to about 50 nm.

In some embodiments, a polynucleotide encodes a nanoparticle as disclosed herein that can include regulatory elements, promoters, enhancer and the like, for expression and/or replication. In some embodiments, a vector as disclosed herein, comprises, or alternatively consists essentially of, or yet further consists of a nanoparticle as disclosed herein.

Also provided is a host cell that comprises, or alternatively consists essentially of, or yet further consists of a virus, VLP, nanoparticle, vector or polynucleotide as disclosed herein. In one aspect, the vector is a plasmid. In one aspect, the host cell is a prokaryotic cell. In another aspect, the host cell is a eukaryotic cell. In one particular aspect, the host cell is a plant cell or a bacterium.

### Compositions

In another aspect, provided herein is a composition comprising, consisting essentially of, or consisting of the combination of formulations comprising a virus, VLP, nanoparticle, polynucleotide, or host cell as provided herein, and at least one carrier, such as a pharmaceutically acceptable carrier or excipient. In one aspect, the composition further comprises a preservative or stabilizer.

In one embodiment, this technology relates to a composition comprising a combination of nanoparticles or formulations as described herein and a carrier.

In another embodiment, this technology relates to a pharmaceutical composition comprising a combination of virus, VLP, nanoparticles or formulations as described herein and a pharmaceutically acceptable carrier.

In another embodiment, this technology relates to a pharmaceutical composition comprising an effective amount or a therapeutically effective amount of a combination of virus, VLP, nanoparticle formulations as described herein and a pharmaceutically acceptable carrier.

Compositions, including pharmaceutical compositions comprising, consisting essentially of, or consisting of the nanoparticle formulation alone or in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins, 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL® PH102, AVICEL® PH105, ELCEMA® P100, EMCOCEL®, VIVACEL®, MING TIA®and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the compositions are administered to a subject suffering from a condition as disclosed herein, such as a human, either alone or as part of a pharmaceutically acceptable formulation, once a week, once a day, twice a day, three times a day, or four times a day, or even more frequently.

Administration of the virus, VLP, VLPs or nanoparticle formulation alone or in combination with the additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

The compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

### Therapeutic Methods

Further disclosed herein are methods for inducing an immune response in a subject consisting essentially of, or yet further consisting of the virus, VLP or nanoparticles, polynucleotides, vectors and/or host cells as disclosed herein.

Also provided are methods for targeting the tumor microenvironment to reverse immunosuppression of a tumor that optionally secretes S100A, the methods comprising, or consisting essentially of, or consisting of contacting the tumor microenvironment with the virus, VLP, nanoparticle, polynucleotide, vector, the composition and/or the host cell of this disclosure. The contacting can be in vitro or in vivo. The S100A9 target can be secreted into the tumor microenvironment, but in one aspect, not necessarily expressed on the surface of tumor cells. Targeting the tumor microenvironment will interact and contact immune cells to reverse immunosuppression and launch anti-tumor immunity through activation of the innate and then adaptive immune system.

Further disclosed herein are methods for treating cancer in a subject in need thereof, comprising, or alternatively consisting essentially of, or yet further consisting of administering to the subject the virus, VLP, nanoparticles, polynucleotides, vectors and/or host cells as disclosed herein.

Further disclosed herein are methods for altering an immune cell profile in lungs of a subject comprising, or alternatively consisting essentially of, or yet further consisting of virus, VLP, nanoparticles, polynucleotides, vectors and/or host cells as disclosed herein.

In some embodiments, a subject is a mammal. In some embodiments, a subject is a human. In some embodiments, a subject has a condition. In some embodiments, a subject has cancer. In some embodiments, a cancer is selected from melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer. In some embodiments, the cancer is primary or metastatic cancer. In some embodiments, the cancer is metastatic or primary lung cancer or breast cancer. In some embodiments, the cancer metastatic melanoma or metastatic triple negative breast cancer. In some embodiments, the cancer is a primary or metastatic cancer in lung. In some embodiments, the cancer expresses or secretes S100A9.

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, or inhalation. In some embodiments, administering is intravenous.

The methods and compositions disclosed herein may further comprise or alternatively consist essentially of, or yet further consists of administering to the subject an anti-tumor therapy other than the virus, VLP, nanoparticle disclosed herein. In some embodiments, anti-tumor therapy may include different cancer therapy or tumor resection. The additional therapeutic can be combined in the same composition or separately administered.

In some embodiments, the nanoparticle and/or composition are provided to prevent the symptoms of cancer from occurring in a subject that is predisposed or does not yet display symptoms of the cancer.

In some embodiments, the virus, VLP, polynucleotide, nanoparticle, vector, or composition disclosed herein may be delivered or administered into a cavity formed by the resection of tumor tissue (i.e. intracavity delivery) or directly into a tumor prior to resection (i.e. intratumoral delivery). In some embodiments. In some embodiments, the administering is intravenous.

In some embodiments, any of the virus, VLP, polynucleotides, nanoparticles, vectors, or compositions disclosed herein, are administered to the subject at least 1, 2, 3, 4, 5, 6, 7, 8, 9, or 10 times a day. In some embodiments, any of polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the virus, VLP, polynucleotides, nanoparticles, vectors, or compositions disclosed herein are administered to the subject at least every 1, 2, 3, 4, 5, 6, 7, 8, 9, or 10 days. In some embodiments, any of the virus, VLP, polynucleotides, nanoparticles, vectors, or compositions disclosed herein are administered to the subject at least every 1, 2, 3, 4, 5, 6, 7, 8, 9, 10, 11, 12, 13, or 14 weeks. In some embodiments, any of the virus, VLP, polynucleotides, nanoparticles, vectors, or compositions disclosed herein are administered to the subject for a period of at least 1, 2, 3, 4, 5, 6, 7, 8, 9, or 10 days. In some embodiments, any of the virus, VLP, polynucleotides, nanoparticles, vectors, or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the virus, VLP, polynucleotides, nanoparticles, vectors, or compositions disclosed herein are administered to the subject for a period of at least 1, 2, 3, 4, 5, 6, 7, 8, 9, 10, 12, 13, 14, 15, 16, 17, 18, 19, or 20 months.

In some embodiments, the method and compositions provided herein, comprising, or alternatively consisting essentially of, or yet further consisting inhibiting metastatic potential of the cancer, reduction in tumor size, a reduction in tumor burden, longer progression free survival, or longer overall survival of the subject.

In one aspect, the methods or compositions further comprise administration of an additional therapeutic agent. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the virus, VLP, nanoparticle with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the additional therapeutic agent comprise, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol -Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, TLR1, TLR2, TLR3, TLR4, TLR5, TLR6, TLR7, TLR8, or TLR9. In some cases, the TLR ligand comprise, or consists essentially of, or yet further consists of, a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the VLP formulation described herein is administered in combination with a radiation therapy.

### Kits

In one particular aspect, the present disclosure provides kits for performing the methods of this disclosure as well as instructions for carrying out the methods of the present disclosure. The kit comprises, or alternatively consists essentially of, or yet further consists of one or more of virus, VLP, nanoparticle, polynucleotide, vector and/or host cell of this disclosure and instructions for use. In a further aspect, the instruction for use provide directions to conduct any of the methods disclosed herein.

The kits are useful for detecting the presence of cancer such as lung cancer in a biological sample e.g., any bodily fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Materials and Methods

### Materials and Cells

RPMI-1640 medium, Hank's Balanced Salt Solution (HBSS), Dulbecco's Modified Eagle's Medium (DMEM), and phosphate buffered saline (PBS) were purchased from Corning Life Sciences. Fetal bovine serum (FBS) was purchased from Atlanta Biologicals. Bovine serum albumin (BSA) was purchased from Sigma-Aldrich. Penicillin/streptomycin, potassium phosphate monobasic and dibasic anhydrous powders, 4-(2-hydroxyethyl)-1-piperazineethanesulfonic acid (HEPES) buffer, sodium acetate anhydrous (NaOAc), methanol, glacial acetic acid, and Sulfo-Cyanine5 (Cy5)-N-hydroxysuccinimide (NHS) esters were purchased from Thermo Fisher Scientific. Dimethyl sulfoxide (DMSO), maleimide-polyethylene glycol8-succinimidyl ester (SM(PEG)₈), sucrose, 10% (v/v) neutral-buffered formalin solution, ethylenediaminetetraacetic acid (EDTA), and Bouin's solution were purchased from Sigma-Aldrich. D-luciferin potassium salt was purchased from Gold Biotechnologies. Ethanol (EtOH) was purchased from VWR International. Paraformaldehyde was purchased from Electron Microscopy Sciences.

Mouse 4T1-Luc (CRL-2539-LUC2) and B16F10 (CRL-6475) cells were purchased from ATCC. 4T1-Luc and B16F10 cells were passaged and grown in RPMI-1640 and DMEM respectively and supplemented with 10% (v/v) fetal bovine serum and 1% (v/v) penicillin/streptomycin. The cells were incubated at 37° C. in a 5% CO2 chamber. RAW-Blue™ Cells (Invivogen, San Diego, CA) were maintained in selection media containing Zeocin (Invivogen) and Normocin (Invivogen) as per instructions by the supplier.

### Preparation of Fluorescent-Labeled and S100A9-Targeted CPMV and Cowpea Chlorotic Mosaic Virus (CCMV)

CPMV and CCMV nanoparticles were propagated in black eyed pea plants and purified as reported in previous work.^{35,67} CPMV was kept at 10 mM potassium phosphate (KP) buffer (pH 7.0-7.2) to the concentration of 2 mg/mL while CCMV was kept in 10 mM NaOAc and 1 mM EDTA at pH 4.8 (from here on out called Buffer B).

SM(PEG)₈ (5 equivalents) dissolved in DMSO was added to the CPMV particle solution and mixed at room temperature (RT) for 2 hours. The solution was ultracentrifuged at 4° C. at 52000 g for 1 hour with a 40% sucrose cushion. The resulting pellet was resuspended in 10 mM KP, and 0.5 equivalents of H6 (MEWSLEKGYTIKGGGSC) or G3 (WGWSLSHGYQVKGGGSC) peptides were added and mixed at RT for 2 hours.22 The solution was then dialyzed using a porous membrane tubing (12-14 kDa, Spectrum Labs) at RT overnight in 10 mM KP to remove unconjugated peptides.

CCMV nanoparticles were diluted to 2 mg/mL in 0.1 M HEPES buffer (pH 7.2). SM(PEG)₈ (5 equivalents) was added and allowed to incubate at RT for 2 hours. The buffer was exchanged to Buffer B using a 10 kDa molecular weight cut off (MWCO) filter, and the resuspended pellet was allowed to sit at RT for 2 hours. The solution was ultracentrifuged at 52000 g for 1 hour, resuspended in Buffer B, and diluted with 0.1 M HEPES. A half equivalent of the corresponding peptide (H6 or G3) was then added and mixed at RT for 2 hours. The buffer was exchanged once more and pelleted with ultracentrifugation as before. The final pellet was resuspended in Buffer B.

To prepare fluorescent CPMV, CPMV particles were diluted to 4 mg/mL and an equal number of equivalents of sulfo-Cy5-NHS esters and SM(PEG)₈ were added. The particles were incubated for 2 hours at RT shielded from light and centrifuged using a 100 kDa MWCO filter for 10-12 minutes at 14000 g. The pellet was resuspended with 5 mM KP buffer and H6 and G3 peptides were added (0.5 equivalents) to the solution. The solution was mixed at RT for 2 hours on an orbital shaker. The solution was then dialyzed using a 12-14 kD MWCO molecular porous membrane tubing (Spectrum Labs) at RT overnight in 10 mM KP.

CCMV fluorescent particles were diluted to 2 mg/mL in 0.1M HEPES (pH 7.2) buffer. Equal number of equivalents of SM(PEG)₈ and sulfo-Cy5-NHS esters were added, and the mixture was incubated at RT away from light for 2 hours. The buffer was then exchanged to Buffer B using a 10 kDa MWCO filter and kept in Buffer B for 2 hours at RT before pelleting down through ultracentrifugation at 52000 g for 1 hour. The particles were resuspended in Buffer B and diluted with 0.1M HEPES before adding the H6 and G3 peptides (0.5 equivalents). The resulting solution was mixed at RT away from light for 2 hours. The buffer was exchanged once more and pelleted with ultracentrifugation as before. The final pellet was resuspended in Buffer B.

### Particle Characterization

### Denaturing Gel-Electrophoresis (SDS-PAGE)

CPMV and CCMV samples were diluted in 100 mM KP or 10 mM Buffer B, respectively, and loaded with 4×LDS Sample Buffer (Life Technologies) for a final concentration of 10 μg in 24 μL. The particles were then denatured at 95° C. for 5 minutes and loaded onto a 12% NuPAGE gel (ThermoFisher Scientific) and ran at 200 V, 120 mA, and 25 W for 40 minutes in 1× morpholinepropanesulfonic acid (MOPS) buffer (ThermoFisher Scientific). The gels were first destained in a mixture of deionized (DI) water, methanol, and acetic acid (50:40:10; v/v) for 30 minutes followed by staining in 0.25% (wt/vol) Coommassie Blue solution for 30 minutes before imaging with the AlphaImager system (Protein Simple).

### Agarose Gel Electrophoresis

CPMV particles were diluted in 100 mM KP; CCMV particles were diluted in 10 mM Buffer B. 6× Gel Loading Purple dye (Biolabs) was added to the CPMV samples. Instead of lithium dodecyl sulfate, glycerol (3 μL) was added to CCMV, and 5 μg of the virus particles were loaded onto a 0.8% (w/v; for CPMV) or 1% (w/v; for CCMV) agarose gel. With CCMV, the gels were run at 4° C. The agarose gel was stained with 1 μL of GelRed Nucleic Acid Gel Stain (Gold Biotechnologies) and run for 30 minutes at 120 V and 400 mA. Immediately after the run, the gel was imaged using the AlphaImager system (Protein Simple) under UV light and then imaged again after staining with 0.25% (wt/vol) Coomassie Blue.

### Dynamic Light Scattering (DLS)

A Zetasizer Nano ZSP/Zen5600 (Malvern Panalytical) was used for DLS measurements, and the CPMV and CCMV particles were diluted to 0.5 mg/mL in 10 mM KP and 10 mM Buffer B respectively. The particles were run at 25° C. with 3 measurements per sample. Zeta potential measurements were carried out using the Smolvchowski method.

### Fast Protein Liquid Chromatography (FPLC)

CPMV and CCMV were diluted to 0.1 mg/mL in 10 mM KP buffer or 10 mM Buffer B and run through a Superose 6 size-exclusion column at 0.5 mL/min for a total volume of 50 mL in an ÄKTA Explorer FPLC machine (GE Healthcare LifeSciences). The elution profile was isocratic, and the UV detectors were fixed at 260 (nucleic acid) and 280 nm (protein).

### Ultraviolet-Visible Spectroscopy (UV-VIS)

UV-VIS (Nanodrop 2000™) was used to calculate the number of fluorescent dyes attached per particle as well as the concentration of VNPs in the solutions. The fluorescent CPMV and CCMV particles were diluted in 0.1 mM KP and 10 mM Buffer B respectively and measured at 260, 280, and 647 nm to calculate the number of conjugated Cy5 particles per VNP. Concentration of the VNP solutions were carried out using the 260 nm wavelength readings. The extinction coefficients of CPMV, CCMV, and the Cy5 dye are 8.1 mL mg⁻¹ cm⁻¹, 5.85 mL mg⁻¹ cm⁻¹, and 270,000 cm⁻¹ M⁻¹, respectively.

### Transmission Electron Microscopy (TEM)

The CPMV and CCMV samples were imaged using a FEI Tecnai Spirit G2 BioTWIN TEM. The samples were loaded onto Formvar carbon film coated TEM supports with 400-mesh hexagonal copper grids (VWR International) at concentrations ranging from 0.25-1 mg/mL in DI H₂O for 2 min. The grids were washed with DI H₂O twice for 45 seconds and then stained with 2% uranyl acetate (Agar Scientific) for 30 seconds twice. The samples were imaged at 300 kV.

### Biodistribution of CPMV and CCMV Particles

All animals were purchased from The Jackson Laboratory and were housed at the Moores Cancer Center (MCC) at the University of California, San Diego (UC San Diego). The animals were granted unlimited access to food and water, and all protocols and studies were compliant with the guidelines set out by the Institutional Animal Care and Use Committee (IACUC) of UC San Diego.

Healthy and B16F10 metastatic tumor-bearing C57BL/6 female mice were used for biodistribution studies of the Cy5-labeled CPMV and CCMV from Section 2. B16F10 cells (200,000 cells/mouse) were administered i.v. and the tumors were matured for one week. The mice were injected with PBS, CPMV-Cy5, CPMV-H6-Cy5, CPMV-G3-Cy5, CCMV-Cy5, CCMV-H6-Cy5, and CCMV-G3-Cy5 i.v. (n=3, 200 μg). After 24 hours, the lungs were harvested and then imaged and quantified for fluorescence using the IVIS (Xenogen).

Further confocal imaging (Nikon AIR Confocal/TIRF STORM microscope) of B16F10-inoculated mice lungs was accomplished using CPMV-Cy5-PEG and CPMV-Cy5-G3 particles (20 mg/kg). After 6 h, mice were sacrificed and briefly perfused with 10 ml of PBS. The harvested lungs were embedded in OCT medium (Fisher Healthcare) and frozen using liquid nitrogen. They were sliced into 10 μm thick sections and mounted on microscope glass slides for immunofluorescence staining. OCT residue was removed using PBS. The tissue sections were blocked with 10% (w/v) bovine serum albumin (BSA) in PBS for 1 h and washed with PBS. Staining was accomplished with a-S100A9 (1:100 dilution, R&D systems, AF2065) and fluorescently-labeled secondary PE a-goat IgG (1:20 dilution) antibodies prepared in 1% (v/v) BSA. The stained tissue samples were mounted on Fluoroshield™ with DAPI for confocal microscopy.

### B16F10 i.v. Challenge to CPMV and CCMV Pre-Treated Mice (Prophylaxis)

C57BL/6 female mice were first treated by i.v. administration of 200 μg of CPMV, CPMV-H6, CPMV-G3, CCMV, CCMV-G3, G3 peptide only, or PBS (n=5). Total free peptide molecules was normalized based on the peptides displayed per CPMV (as determined by ImageJ analysis of separated coat proteins on SDS-PAGE gels). After 7 days, the mice were challenged by i.v. administration of 200,000 B16F10 melanoma cells per mouse. Lungs were harvested at day 21 (day 14 after tumor inoculation) and fixed in a 10% (v/v) neutral-buffered formalin solution overnight. Following fixation, the lungs were stored in 70% (v/v) ethanol (EtOH), and the number of tumor nodules per lung was manually counted.

After tumor nodule counting, the fixed lung samples were submitted to the La Jolla Institute for Immunology for hematoxylin and eosin (H&E) staining and imaging. Paraffin-embedded blocks were sectioned at 4 μm on a Leica RM2125 RTS microtome. The sections were then floated on a 42° C. tissue flotation bath and mounted onto Fisher Superfrost Plus microscope slides and subjected to H&E staining. Scanning was accomplished using a ZEISS AxioScan Z1 using a 20× objective. The ratio of tumor cells to total cells was measured from the histology slides using QuPath software⁶⁸.

### 4T1-Luc I.V. Challenge to CPMV Pre-Treated Mice (Prophylaxis)

Balb/c mice were first treated by i.v. administration of 225 μg CPMV-G3, CPMV, or PBS (n=4). After 5 days, the mice were challenged by i.v. injection of 200,000 4T1-Luc cells. The mice were imaged with the IVIS using luminescent imaging every 3 days by injecting intraperitoneally (i.p.) 150 mg/kg of body weight D-luciferin. ROI measurements were taken through the Living Image 3.0 software. The weight of the mice was also tracked every 3 days. After 25 days (20 days after tumor inoculation), the lungs were collected and fixed in Bouin's solution for 3 days. Each tumor nodule on the lungs was counted manually and averaged between the mice in each group.

### Treatment of B16F10 Melanoma and 4T1 Metastasis Using CPMV Particles

B16F10 cells (40,000/mouse) were injected i.v. into female C57B6/J mice (n=7-12). Three days after tumor inoculation, the mice were injected i.v. with PBS, CPMV, CPMV-H6, and H6 (200 μg/mouse). H6 peptide only controls were injected at 20% the number of total CPMV coat proteins as estimated from Image-J analysis of peptide conjugation success. Fifteen days following particle injections, the organs were harvested and stored in 10% neutral buffered formalin solution overnight. The organs were moved to 70% (v/v) EtOH the next day and the tumor nodules were individually counted.

4T1-luc cells (100,000/mouse) were injected i.v. into female Balb/c mice (n=15). Three days following tumor injection, the mice were split into three groups (n=5) consisting of PBS, CPMV, and CPMV-H6 treatments (200 μg/mouse). Tumor growth was monitored by total bioluminescence imaging based on the i.p. injection of 150 mg/kg of body weight D-luciferin. Total bioluminescence was determined using the Living Image 3.0 software, and ROI were quantified as average counts.

### Immunogenicity Profile of CPMV and CCMV Particles in vitro

The immunogenicity of CPMV and CCMV was compared through a RAW-Blue™ assay (Invivogen). Briefly, 100,000 RAW-Blue™ cells/well were incubated with 0.5 μg of CPMV and CCMV, 50 EU mL-1 E. coli endotoxin standard control (ThermoScientific), or culture media for 18 hours. Toll-like receptor (TLR) and nucleotide-binding oligomerization domain (NOD) stimulation was assessed by measuring the levels of secreted embryonic alkaline phosphatase (SEAP) using a QUANTI-Blue™ (Invivogen) assay. Absorbance was measured at 655 nm using a Tecan microplate reader.

The immunogenicity between CPMV and its peptide-conjugated counterparts was also assessed using a RAW-Blue™ assay. RAW-Blue™ cells were incubated with 10 μg of CPMV, CPMV-H6, CPMV-G3, lipopolysaccharide (LPS), and H6 and G3 peptide for 24 hours. A QUANTI-Blue™ assay was run like before, and absorbance was measured at 655 nm.

### Flow Cytometry for Innate Immune Cell Profile In Vivo

C57BL/6 mice were treated i.v. using CPMV, CPMV-H6, CPMV-G3, or PBS (n=3) at a dose of 200 μg per mouse. Lungs were harvested after 24 hours. Harvested organs were each placed in separate gentle MACS C tubes (Miltenyi Biotec) and dissociated with enzymatic solutions (lung dissociation kits, Miltenyi Biotec). The C tube lid was tightly screwed, inverted, and inserted in a gentleMACS dissociator (Militenyi Biotec). Organs were minced and digested on appropriate gentleMACS programs. After digestion, cells were passed through 40 μm pore size strainers and centrifuged at 500 g for 5 min. The cell pellet was resuspended in 5 mL of 1× RBC lysis solution (eBioscience) and incubated at RT for 2 min, followed by the addition of RPMI-1640 medium to stop lysis. Cells were centrifuged again at 500 g for 5 min and resuspended in PBS with 1% BSA and 2 mM EDTA (FACS solution). Total cell count and viability were found using trypan blue solution (Sigma-Aldrich) and a Countess™ automated cell counter (Invitrogen). Cell concentrations were adjusted to 1.0×10⁷ cells/mL.

To reduce background staining of antibodies, 0.25 μg of purified rat anti-mouse CD16/32 (Biolegend) was added to 100 μL cell suspensions before adding to the conjugated antibodies. Cells were incubated at RT for ten minutes prior to immunofluorescence staining. All antibodies for immunofluorescence staining were purchased from Biolegend and diluted in FACS solution according to the manufacturer's recommendation. The markers used were for dendritic cells (DC) (CD11b, [M1/70]; CD11c, [N418]), activated DCs (DC markers plus MHCII, [M5/114.15.2]; CD86, [GL-1]); macrophages (CD11b, [M1/70]; Ly6G-F4/80, [1A8]), M1 macrophages (macrophage markers plus MHCII, [M5/114.15.2]; CD86, [GL-1]), and neutrophils (Ly6G, [1A8]; Ly6C, [HK1.4]; CD11b, [M1/70]). The protocol is as follows: 50 μL of antibody solution was added to each well in a 96 well plate, followed by the addition of 100 μL of cell suspension. Cells were incubated at RT for 30 min in the dark. After 30 min, cells were centrifuged at 500 g for 5 min, the supernatant was aspirated, and the plate was vortexed to loosen cells. Cells were washed by adding 300 μL of FACS solution and repeating centrifugation. After staining, cells were fixed using 2% (w/v) paraformaldehyde (PFA) in PBS for 15 min, washed once, and resuspend in 200 μL FACS solution. The cells were stored at 4° C. overnight prior to measurement. BD FACScelesta (BD Bioscience) and FlowJo were used for data acquisition and analysis, respectively.

### Aspartate Aminotransferase (AST) and Alanine Aminotransferase (ALT) Liver Toxicity Assays

C57BL/6J mice (n=5-7) were treated i.v. with CPMV, CPMV-H6, CPMV-G3, H6, G3, or PBS (200 μg CPMV/mouse; the peptide dose was normalized to match the number of peptides delivered by CPMV). After 1, 3, and 7 days, blood was collected through retroorbital bleeds using heparinized tubes (Fisher Scientific). The blood was spun down at 7,500 rpm for 10 min at 4° C. and the sera was collected and stored at −80° C. The sera were then subjected to AST and ALT activity testing by following the manufacturer's guidelines (Abcam). Briefly, the sera were diluted 10×, and compared against a standard curve of pyruvate and glutamate for the ALT and AST assay, respectively. Fluorometric readings at 535 nm (excitation) and 587 nm (emission) and at 10 and 40 min were used to measure ALT activity while AST activity was measured using absorbance readings at 450 nm also at 10 and 40 min (Tecan plate reader).

### Statistical Analysis

All figures and data analysis were created and accomplished using Prism 5 (GraphPad Software). All statistical significance was determined using either one or two-way analysis of variance (ANOVA). Statistical significance from Kaplan-Meier plots was analyzed using Mantel-Cox tests.

### S100A9-Targeted Plant Virus Nanoparticles

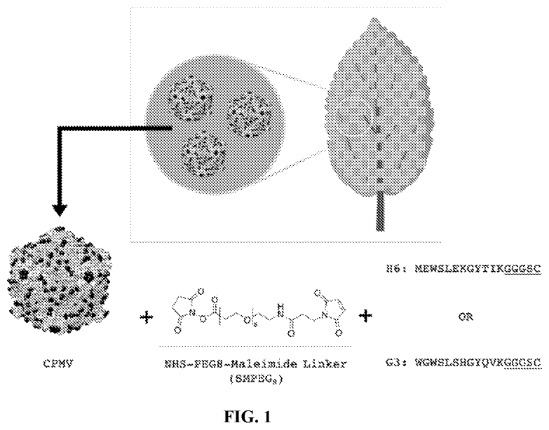

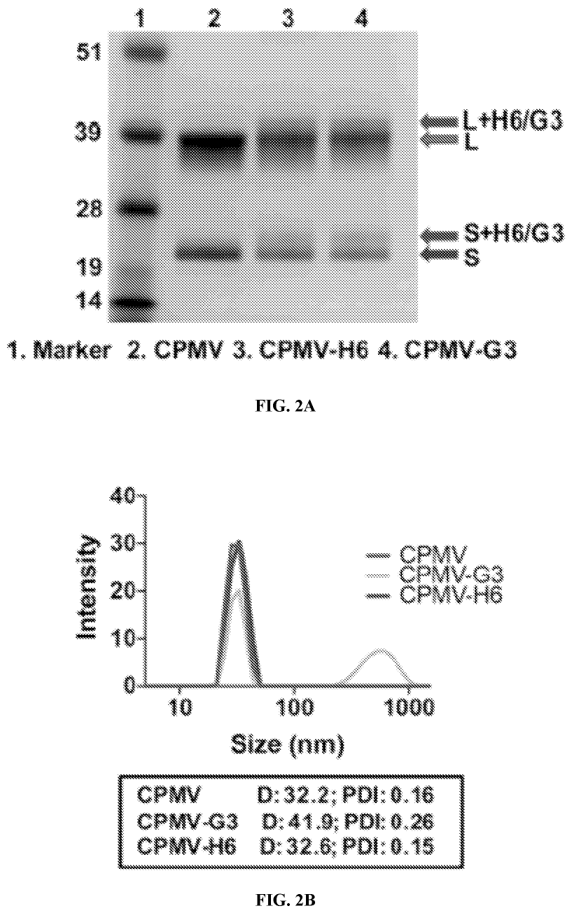

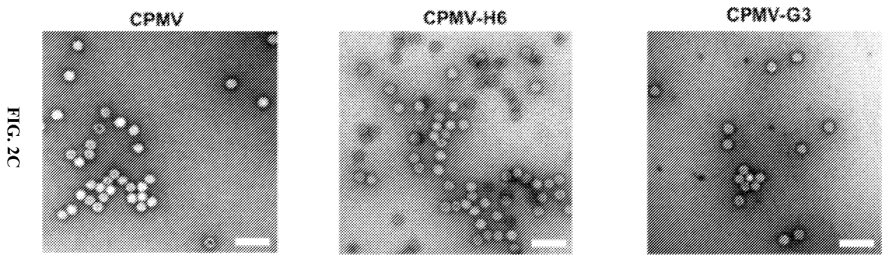

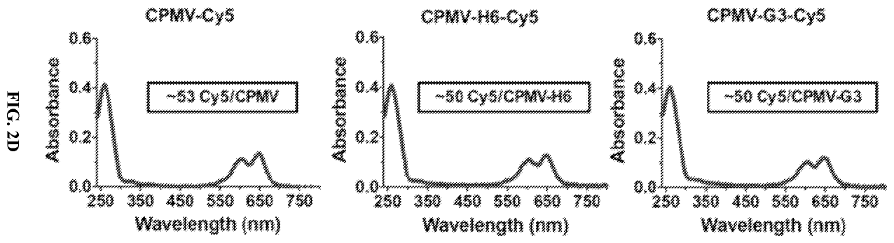

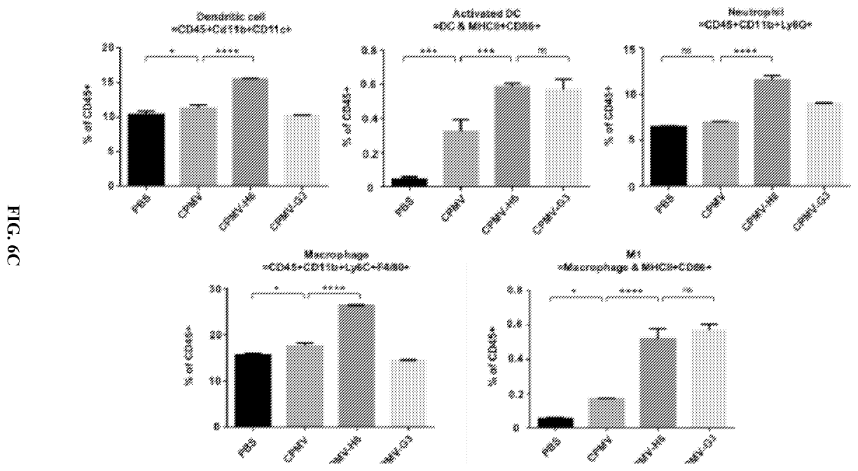

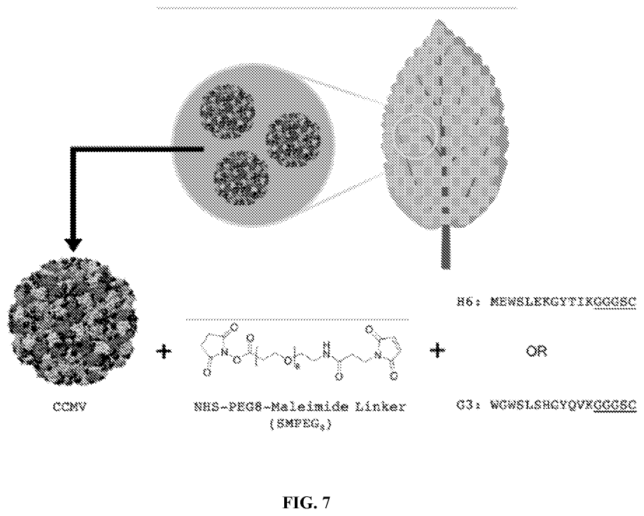

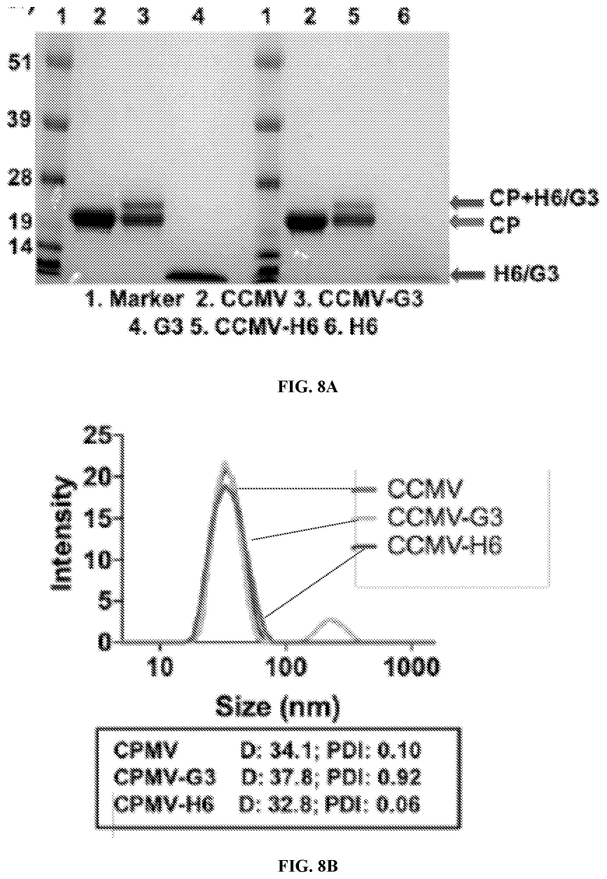

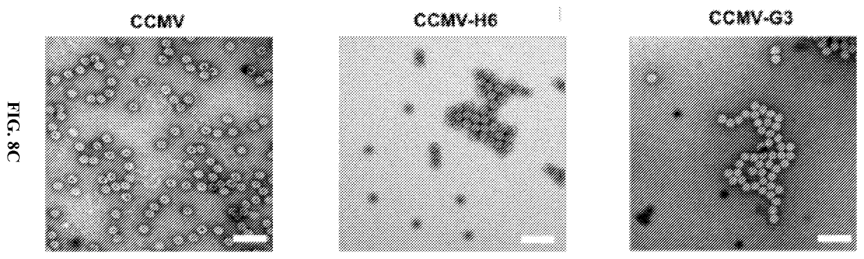

CPMV and cowpea chlorotic mottle virus (CCMV) control nanoparticles were purified from infected black-eyed pea No. 5 plants. The CCMV nanoparticles served as a control because unlike CPMV, the CCMV nanoparticles do not elicit anti-tumor immunity when used as in situ vaccine.³² H6 and G3 peptides were synthesized with a C-terminal GGGSC linker for conjugation to the viral nanoparticles (VNPs), which offer solvent-exposed lysine side chains.^{33,34} Conjugation was achieved via use of the heterobifunctional linker SMPEG8, where the NHS ester reacts with lysines on CPMV/CCMV and the maleimide reacts with the cysteine on the peptide (FIG. 1 and FIG. 7, respectively). To characterize peptide conjugation, the particles were denatured and the coat proteins (CPs) analyzed by SDS-PAGE (FIG. 2A and FIG. 8A). The capsid of CPMV particles consist of 60 copies each of a large and small CP (42 and 24 kDa, respectively) while CCMV particles consist of 180 copies of one 20 kDa CP. SDS-PAGE confirmed successful conjugation with higher molecular weight bands detectable for the CPs. The molecular weight of the peptides are 1846 and 1809 g/mol for the H6 and G3 peptides, respectively; therefore, the band pattern is consistent with a mosaic of unmodified and peptide-displaying CPs for both the CPMV and CCMV formulations (FIG. 1 and FIG. 7). Band analysis using ImageJ indicated roughly 20% of CPMV and 17% of CCMV CPs were conjugated to the peptide indicating ˜24 and 31 peptides per particle, respectively.

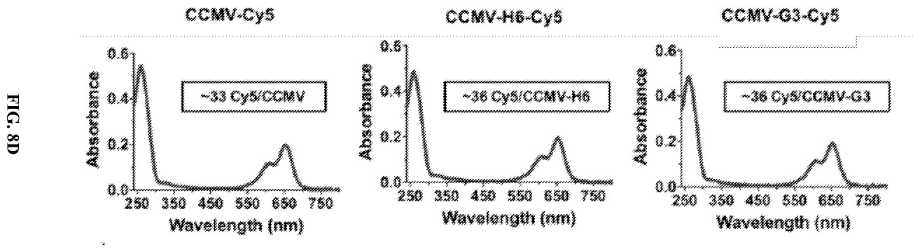

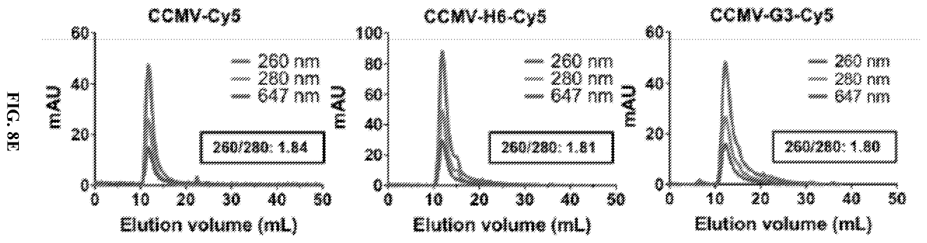

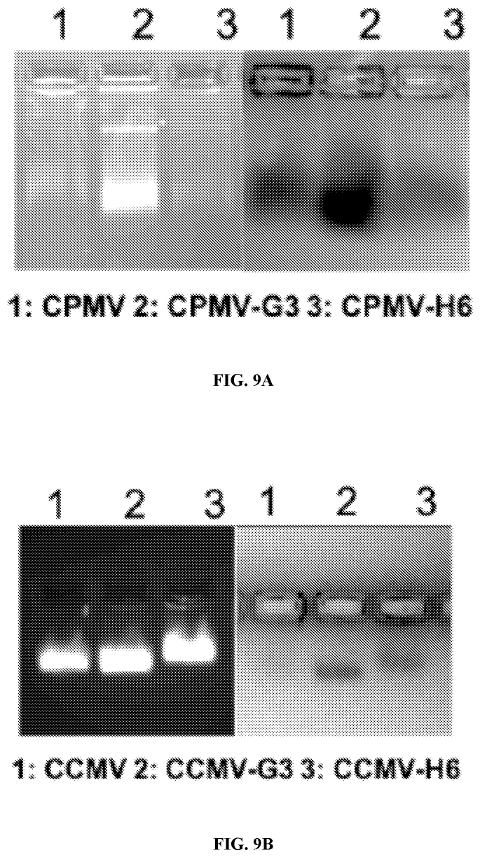

To validate the structural integrity of the S100A9-targeted CPMV and CCMV formulations, native agarose gel electrophoresis, size exclusion chromatography (using FPLC), and TEM imaging was carried out. Native agarose gels on intact VNPs indicate stable capsids with minimal aggregation upon peptide conjugation (FIG. 9A, FIG. 9B). Additional FPLC measurements of the CPMV and CCMV particles show absence of any impurities such as free CP or broken particles with a single peak indicating monodisperse particles (FIG. 9C, FIG. 9D). DLS measurements were consistent with the reported size of CPMV and CCMV^{29,35} and indicate presence of monodisperse nanoparticles with hydrodynamic diameters of approximately 30 nm (FIG. 2B and FIG. 8B). The low polydispersity indices (FIG. 2B and FIG. 8B black box) indicate none to minimal aggregation of the particles after peptide conjugation. The G3-conjugated CPMV and CCMV particles did showcase some level of aggregation although this was not deemed largely significant to warrant exclusion from future studies. Differences in the charge of G3, which is net positive at neutral pH, and H6, which is net neutral, may explain the aggregation of CPMV-G3 and CCMV-G3. Conjugation of the G3 peptide may mask the inherent negative charge of CPMV leading to diminished charge-charge repulsion.³² Regardless, the TEM images show that both the CPMV and CCMV particles are structurally intact, measuring ˜30 nm, an out peptide conjugation (FIG. 2C and FIG. 8C).

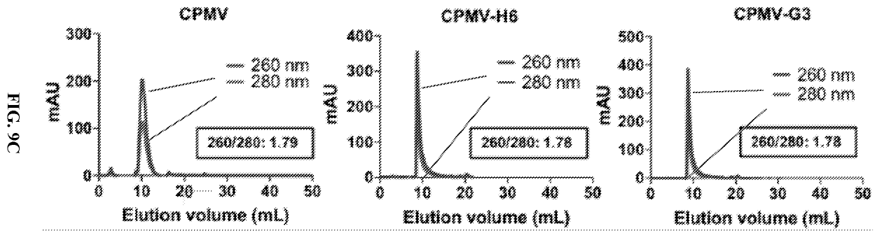

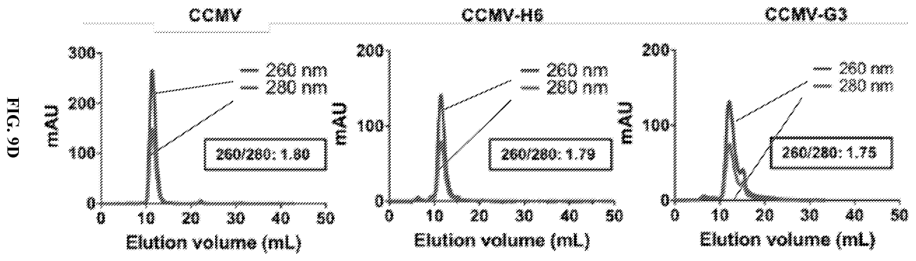

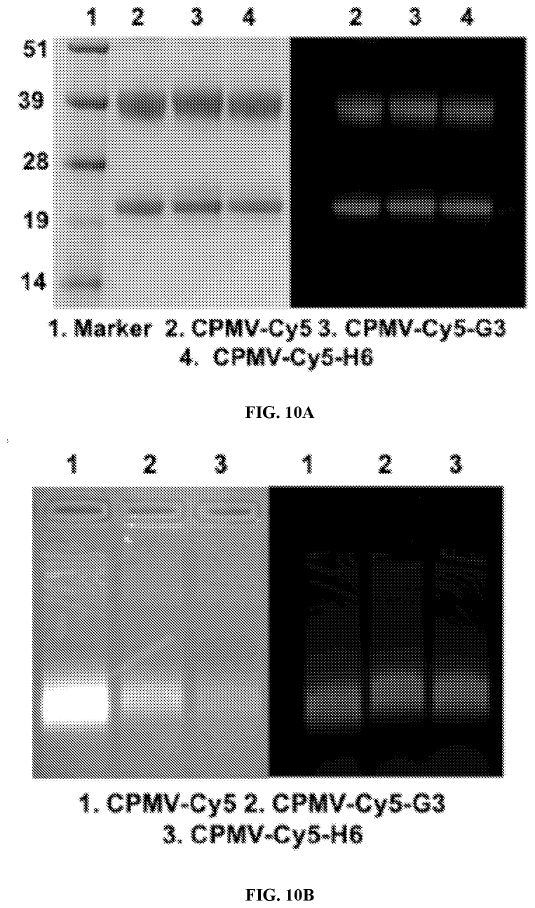

For the biodistribution study, CPMV and CCMV nanoparticles were dual labeled with H6/G3 peptides and Cy5. S100A9-targeted, fluorescent VNPs were analyzed by SDS-PAGE and native agarose gel electrophoresis to confirm that the Cy5 label was covalently introduced—as evident by appearance of fluorescent protein bands (FIG. 10A and FIG. 10B). UV-VIS measurements and the Beer Lambert law were used to determine the number of dyes per particles, and we found consistent labeling with ˜50 Cy5 labels conjugated to CPMV, CPMV-H6, and CPMV-G3 (FIG. 2D). For CCMV, ˜35 Cy5 labels were conjugated to CCMV, CCMV-H6, and CCMV-G3 (FIG. 8D). FPLC analysis was consistent with intact and labeled VNPs being eluted from the column (FIG. 2E and FIG. 8E). The dye co-elutes (absorbance measured at 647) with the RNA and protein signals (measured as 260 nm and 280 nm, respectively) indicating successful conjugation. Also, the absorbance ratio at 260/280 nm is consistent with intact particles; the absorbance ratio of intact CPMV and CCMV is 1.8.

### Biodistribution of CPMV and CCMV Particles

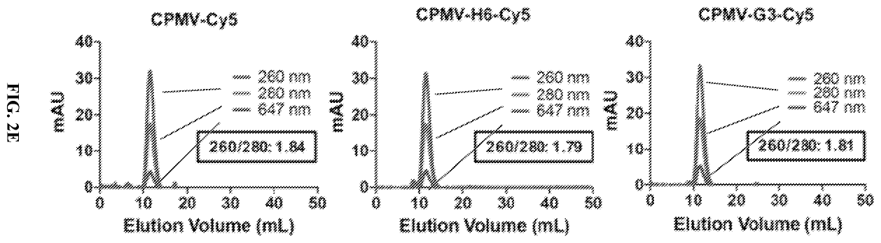

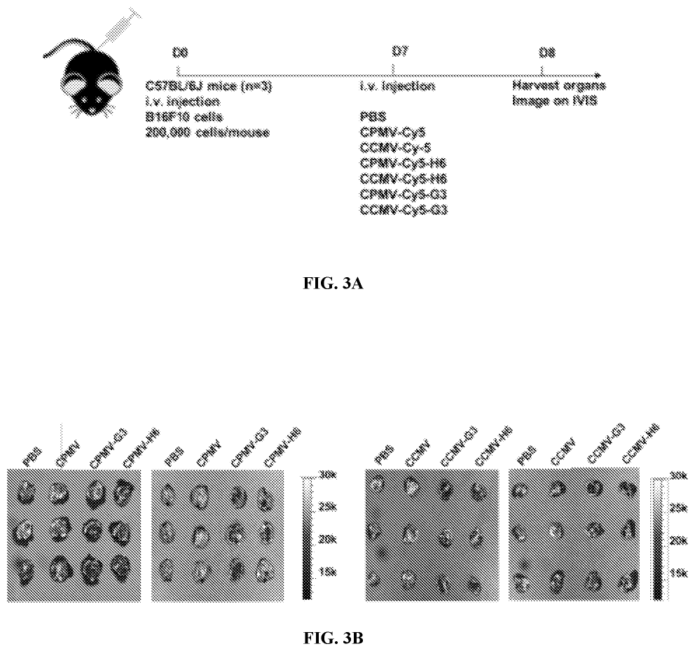

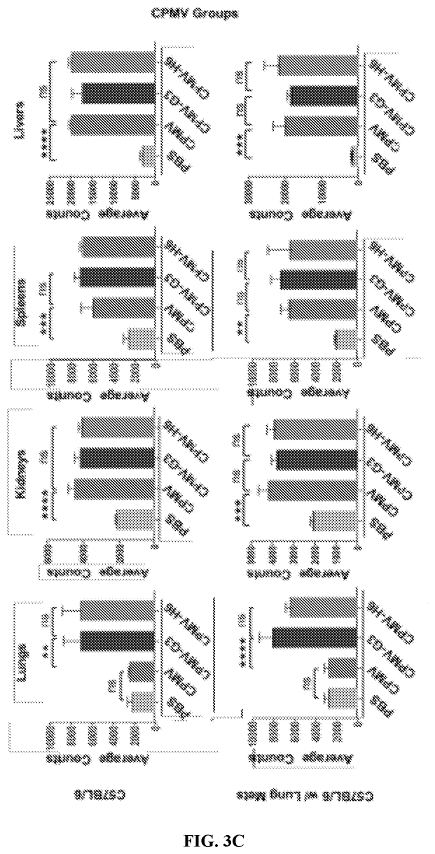

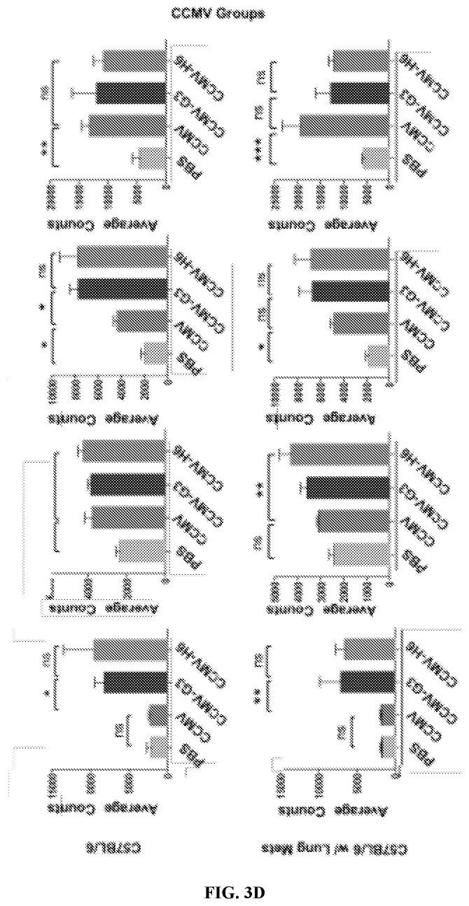

Both healthy and B16F10 tumor-bearing mice were injected with native and H6/G3-conjugated CPMV and CCMV; to enable tracking the nanoparticles were labeled with Cy5. Lungs and other organs were harvested 24 h following intravenous (i.v.) nanoparticle injection and imaged ex vivo to observe localization of the nanoparticles in the lungs, spleen, kidney, and liver (FIG. 3A). Fluorescent imaging and quantitative data analysis indicates that native CPMV and CCMV do not home to the lungs, but are cleared instead in other organs such as the liver (75%) and spleen (14-18%) and to a lesser degree by the kidneys (6-10%) (FIG. 3B, FIG. 3C and, FIG. 11B, FIG. 11C respectively), as previously reported.³⁶ Likewise, the S100A9-targeted CPMV and CCMV nanoparticles accumulated in the liver and spleen, but there was also significant accumulation within the lungs regardless of tumor inoculation.

Approximately 19% of the CPMV-Cy5-G3 and 17% of the CPMV-Cy5-H6 nanoparticles accumulated within the lungs of healthy mice. Lung accumulation within tumor-inoculated mice was similar with 18 and 12% accumulation of the CPMV-Cy5-G3 and CPMV-Cy5-H6, respectively. The lung homing was also reflected by reduced liver clearance, 55-68% for the S100A9-targeted vs. 75% for native CPMV. No changes in spleen or kidney deposition were noted. Overall, the trend was similar for CCMV formulations. While native CCMV does not home to the lungs, H6/G3 conjugation led to significant lung accumulation of up to 35% in healthy mice and 29% in tumor-bearing mice (FIG. 3B, FIG. 3D).

To investigate the co-localization of the CPMV particles with S100A9, confocal imaging of tumor-bearing lungs sections was performed. CPMV particles showed no association with S100A9 (not shown). In contrast, CPMV-Cy5-G3 particles strongly co-localized with S100A9 as indicated by fluorescent overlaps between the CPMV-Cy5-G3 (shown in yellow) and the S100A9 (shown in teal) (FIG. 3E). ImageJ co-localization analysis using the Fiji Coloc2 platform reveals a Mander's M2 colocalization coefficient of 0.58 for CPMV:S100A9 or 0.74 for S100A9:CPMV indicating that indeed there is association of the CPMV-Cy5-G3 and S100A9.

### B16F10 I.V. Challenge to CPMV and CCMV Pre-Exposed Mice (Prophylaxis)

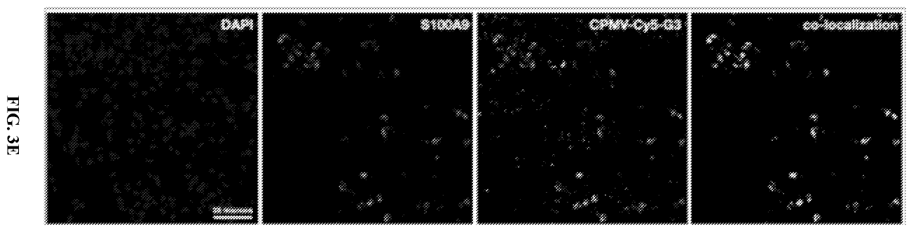

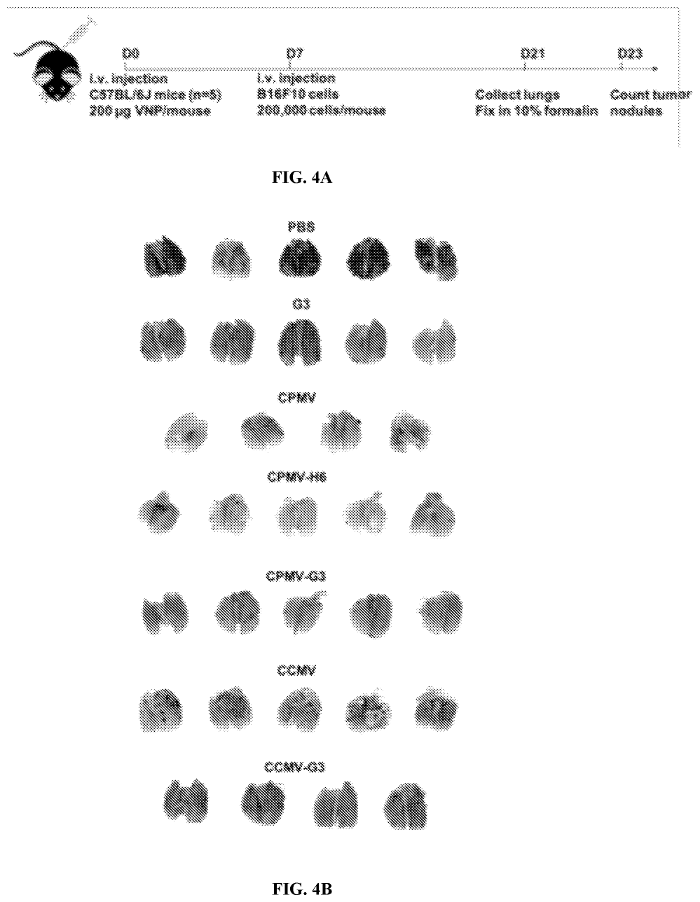

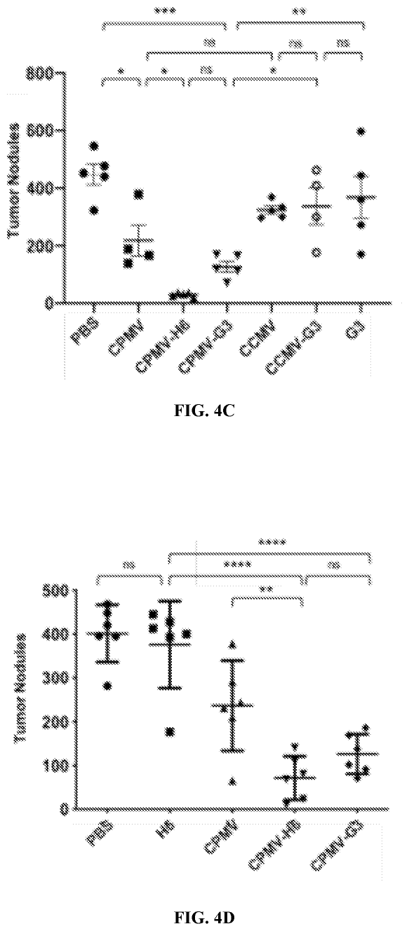

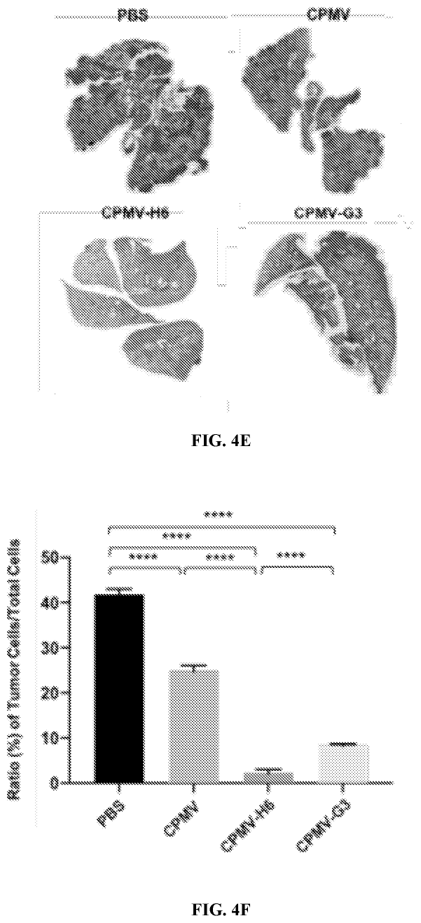

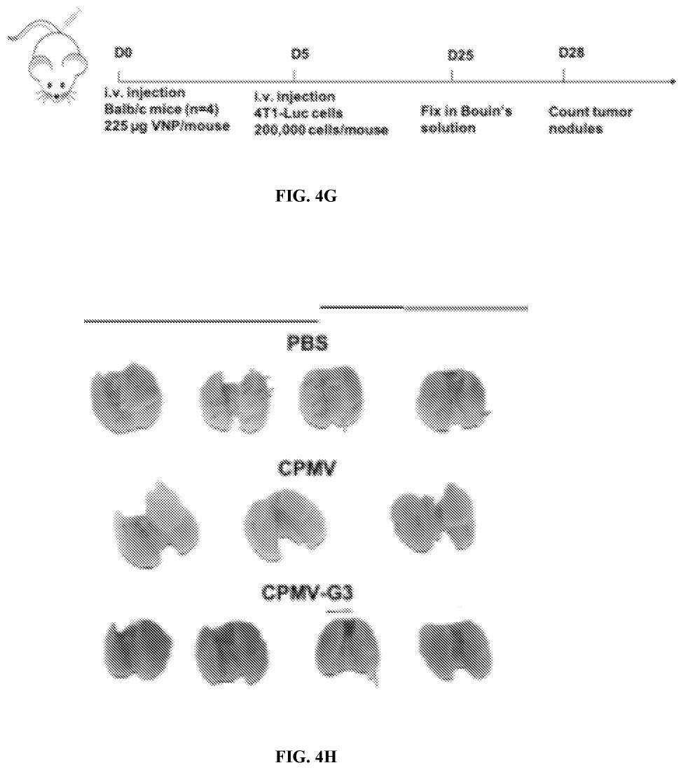

To investigate the suitability of S100A9-targeted VNPs to serve as a prophylactic immunotherapy preventing manifestation of lung metastasis, we used a lung metastasis mouse model using C57BL/6J mice i.v. challenged with B16F10 melanoma cells. C57BL/6J mice were pre-exposed to the CPMV (therapeutic) and CCMV (control) nanoparticles with and without the H6/G3 targeting ligands, one week before being challenged i.v. with B16F10 melanoma cells (FIG. 4A). Lungs were harvested 2 weeks post tumor challenge, and tumor nodules were manually counted. Data demonstrate that lungs harvested from animals treated with CPMV-H6 and CPMV-G3 nanoparticles significantly decreased tumor burden in the lungs by 14.8 (p<0.0001) and 3.5 fold (p=0.0002) compared to the PBS control, respectively (FIG. 4B, FIG. 4C). This effect was not seen with the S100A9-targeted or native CCMV control nanoparticles and the G3 peptide only control. Native CPMV nanoparticles also had a significant effect and treatment resulted in a 2.1 fold (p=0.0168) decrease in formation of tumor nodules compared to the PBS control. This experiment was repeated one more time with only the CPMV particles as well as the H6 peptide only control (FIG. 4D). The repeated experiment produced very similar results with the CPMV-H6 and CPMV-G3 particles showcasing significantly reduced tumor nodules compared to the PBS (5.6 (p<0.0001) and 3.2 fold (p<0.0001), respectively) and H6 peptide only controls (5.2 (p<0.0001) and 3.0 fold (p<0.0001), respectively). The CPMV again showed some level of effectiveness (1.7 fold reduction (p=0.0081) compared to PBS) although it was to a lesser degree than the peptide-conjugated nanoparticles. The CPMV-H6 formulations had a 5-fold enhanced efficacy vs. CPMV (p=0.077) and CPMV-G3 exhibited 2-fold increase in efficacy as compared to CPMV although this was deemed insignificant (p=0.1212).

10267| The lungs of the CPMV mice were further examined through histology and H&E staining (FIG. 4E). Qualitatively, the histology slides exemplify that the CPMV treatment greatly reduces tumor burden. There is a stark decrease in tumor cells (dark purple) indicative of the B16F10 tumor nodules found in the lungs when injected with CPMV-H6 and CPMV-G3 compared to PBS and CPMV. The ratio of tumor cells to total cells was analyzed using QuPath software, which illustrates that the CPMV-H6 and CPMV-G3 treatment quite significantly reduces tumor cell count. CPMV-H6 and CPMV-G3 reduced the ratio of tumor:total cells by 18-fold (p<0.0001) and 5-fold (p<0.0001), respectively. Again, native CPMV displayed efficacy yet at significantly lower levels achieving only 1.7-fold reduction (p<0.0001) (FIG. 4F).

### 4T1-Luc I.V. Challenge to CPMV Pre-Exposed Mice (Prophylaxis)

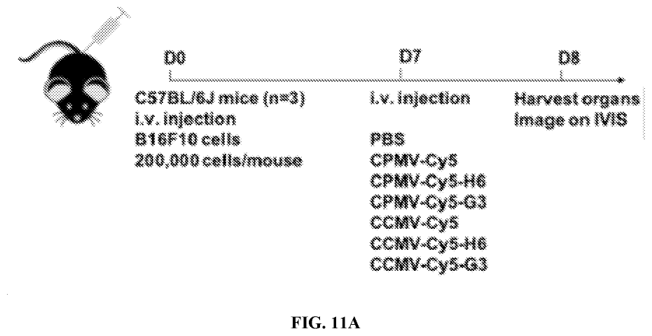

To determine whether the prophylactic effect of S100A9-targeted CPMV could be replicated in other tumor models, we first exposed Balb/C mice to CPMV and S100A9-targeted CPMV by i.v. injection, and then challenged mice with luciferase-labeled 4T1 (4T1-Luc) cells. This experimental lung metastatic model mimics metastatic triple negative breast cancer (TNBC). Tumor cell challenge was carried out 5 days post CPMV exposure (FIG. 4G). Bioluminescent imaging was carried out using the in vivo imaging system (IVIS) imager to track 4T1-Luc cells, and imaging of the PBS treated animals shows that lung metastases established within 2 weeks post tumor cell challenge. By day 21, all the mice in the PBS treatment group had to be sacrificed due to significant weight loss (FIG. 12B). On the contrary, both the CPMV and CPMV-G3 pre-exposed mice showed no signs of tumor growth by day 21. The weight of the mice in both CPMV groups stayed fairly consistent throughout the experiment without any significant loss; no apparent side effects were observed (FIG. 12B). The lungs were harvested after 25 days and fixed in Bouin's solution before manual counting of tumor nodules (FIG. 4H, FIG. 4I). Compared to PBS, the CPMV showed a 10.6-fold decrease in tumor nodules (p=0.0015) while the CPMV-G3 demonstrated a 99-fold decrease (p=0.0005) (FIG. 4H, FIG. 4I). There was no significant difference between the CPMV and the CPMV-G3.

### Investigating S1009-Targeted CPMV as an Immunotherapy After Establishment of Tumors from B16F10 and 4T1-Luc (Immunotherapy)

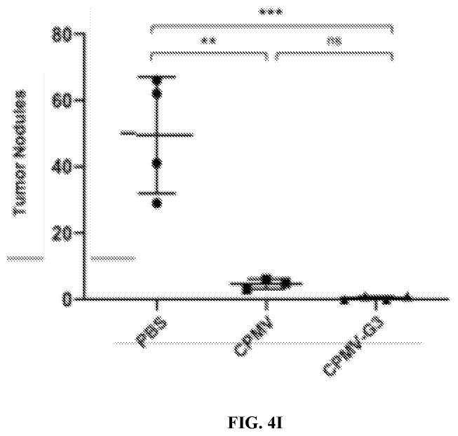

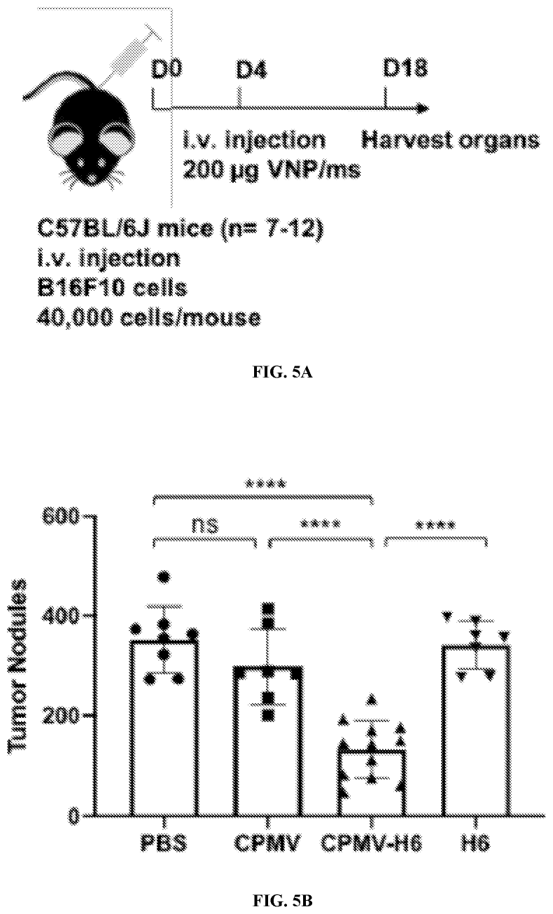

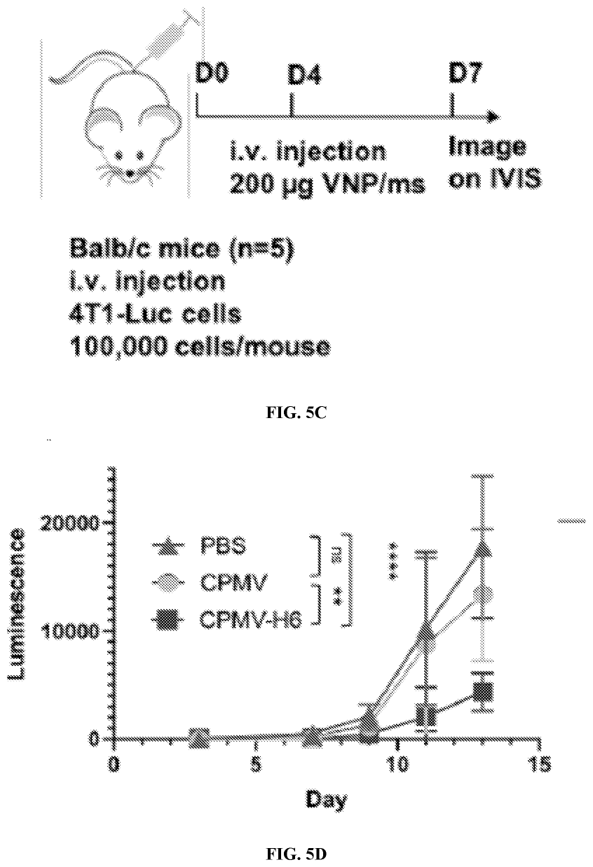

The CPMV particles were additionally tested as a potential immunotherapy and administered in both B16F10 and 4T1-Luc inoculated mice after establishment of the disease. C57BL/6J mice (n=7-12) were inoculated with B16F10 cells and treated with PBS, CPMV, CPMV-H6, and H6 peptide after 4 days (FIG. 5A). After 18 days, the lungs were harvested and the tumor nodules were manually counted. The CPMV-H6 particles as an immunotherapy demonstrated significant advantages compared to all the controls (FIG. 5B). CPMV-H6 administration decreased tumor nodules by 2.7-fold compared to PBS (p<0.0001), 2.3-fold compared to unconjugated CPMV (p<0.0001), and 2.6-fold compared to H6 peptide only (p<0.0001). Unlike in the immunoprophylaxis, the CPMV only control did not show any significant decrease in tumor nodules compared to the PBS only control.

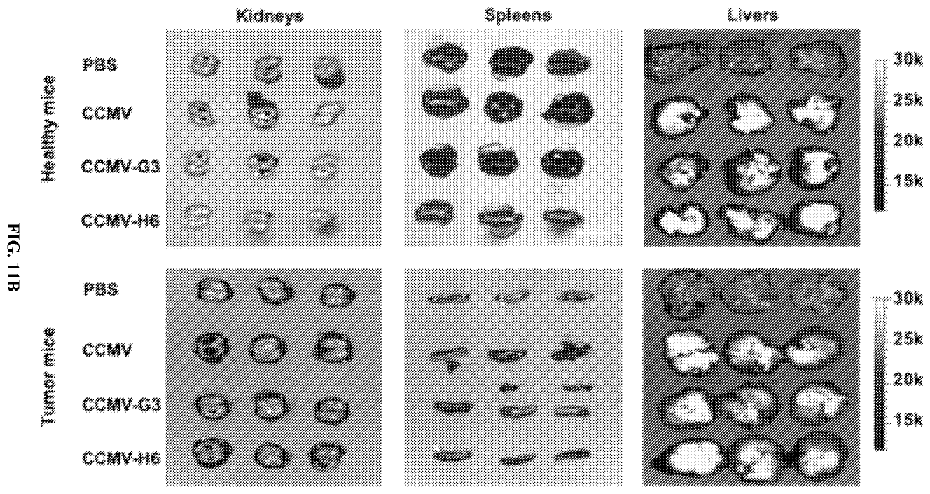

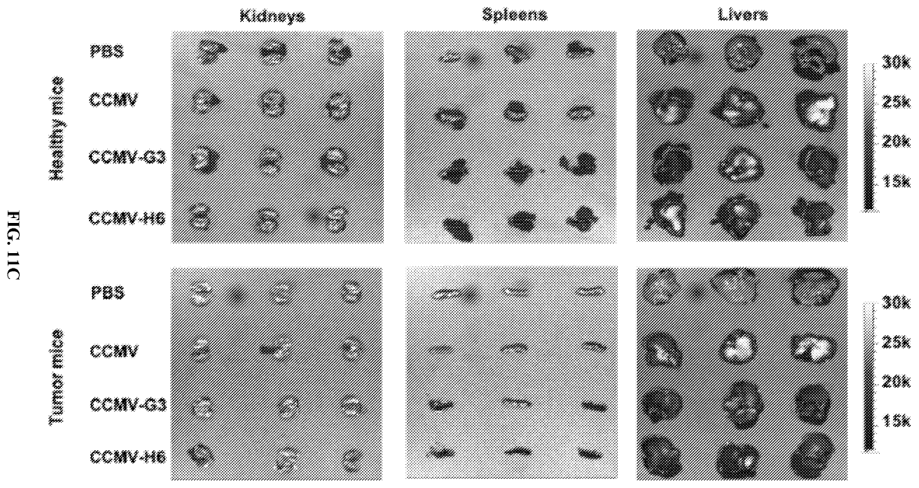

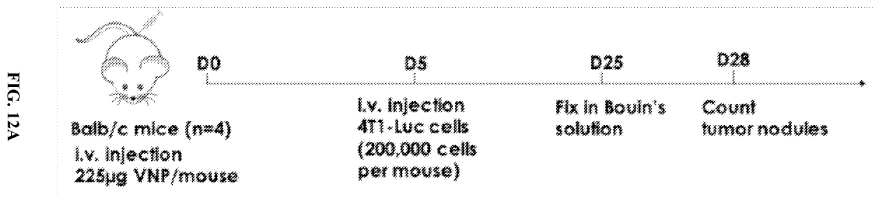

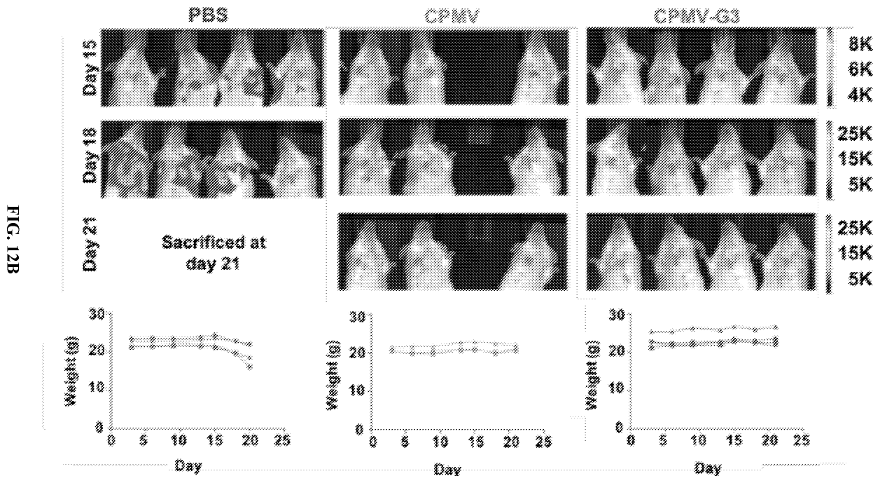

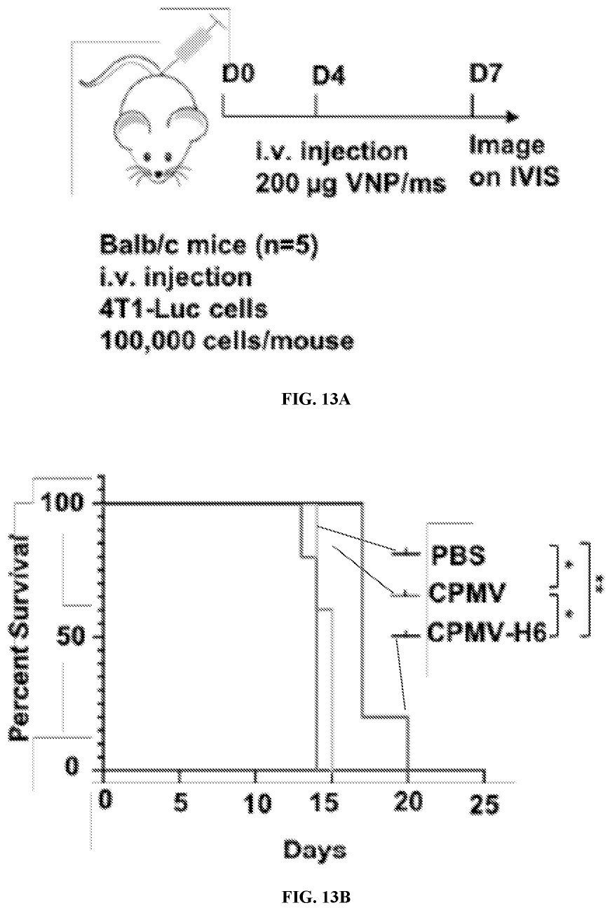

The CPMV-H6 particles were further tested in a 4T1-Luc model by injecting female Balb/c mice (n=5) with the particles 3 days post tumor inoculation (FIG. 5C). Untreated tumors developed quickly and significant tumor burden was observed in the PBS and CPMV treatment groups by day 7 (FIG. 5D, FIG. 5E). A representative IVIS image on day 9 is shown in FIG. 5E while further imaging can be found in FIG. 13. Similar to the prophylactic immunotherapy study, the tumor burden in the lung was less severe and disease progression was delayed in the mice treated with CPMV-H6 (FIG. 13C). The unabated tumor growth led to the mice in the PBS and CPMV groups all dying within 15 days or reaching their clinical endpoints before being sacrificed (FIG. 13B). The CPMV-H6 treatment was able to extend the median time of survival by 3 days while the CPMV treatment extended the median time of survival by 1 day.

### Immunogenicity of CPMV

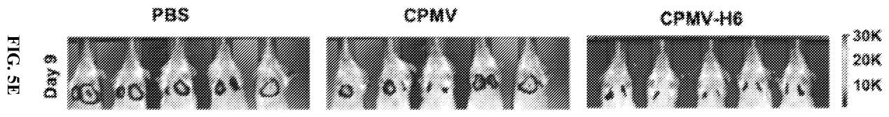

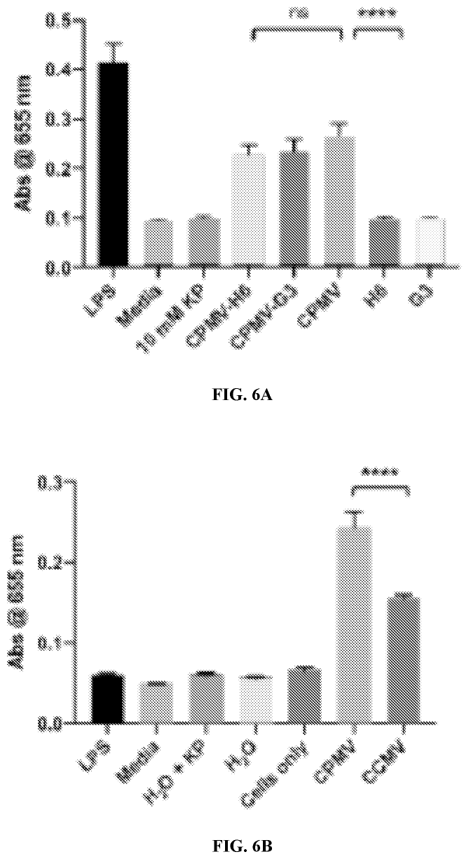

To gain insights into the underlying mechanism, we first evaluated the immunogenicity of targeted and native CPMV vs. CCMV using a RAW-BLUE™ assay (FIG. 6A, FIG. 6B). Following 24 h incubation with the particles and peptides, the wild type CPMV and the peptide-conjugated CPMV exhibited higher level of activation of transcriptional factors (i.e., NF-κB and AP-1) compared to CCMV control nanoparticles. The H6/G3 peptides alone were not immunostimulatory indicating, as expected, the peptides alone are not TLR and NOD agonists. Overall, the immunogenicity assays confirm that CPMV acts as an immunostimulatory adjuvant.

To assay whether CPMV targeting to the lungs would alter the immune cell profiles, lungs were collected 24 h post CPMV treatment and innate immune cell profiles were analyzed using flow cytometry. Indeed, data highlight considerable changes in the immune cell profiles in the lungs 24 h after particle administration (FIG. 6C). While PBS and CPMV did not produce a significant impact, CPMV-H6 treatment in particular led to increased infiltration of leukocytes, especially dendritic cells (DC) and neutrophils. Compared to PBS, CPMV-H6 increased the percentage of DCs in the lungs by 1.5 fold (p<0.0001) and neutrophils by 1.8 fold (p<0.0001). The H6-conjugated particles also improved DC and neutrophil infiltration compared to native CPMV by 1.4 fold (p<0.0001) and 1.7 fold (p<0.0001), respectively. CPMV-G3 did not significantly improve DC recruitment although it did increase neutrophil infiltration by 1.4 (p<0.0001) and 1.3 fold (p<0.0001) compared to PBS and native CPMV, respectively. CPMV-H6 additionally increased macrophage infiltration by 1.7 (p<0.0001) and 1.5 fold (p<0.0001) compared to PBS and CPMV, respectively, although this effect was not observed with CPMV-G3. When observing immune cell activation, CPMV-G3 and CPMV-H6 performed equally with insignificant differences between the two for DC activation and M1 macrophage polarization. However, against the controls, there was a significant improvement of DC activation by CPMV-H6 and CPMV-G3 with 12.5 (p<0.0001) and 12.1 fold (p=0.0006) increases compared to PBS. Both CPMV-H6/G3 increased DC activation by 1.8 fold (p=0.0006 for H6 and p=0.0009 for G3). When comparing M1 activation, CPMV-H6 improved activation by 9.9 (p<0.0001) and 3.1 fold (p<0.0001) compared to PBS and CPMV, respectively while CPMV-G3 similarly improved activation by 10.8 (p<0.0001) and 3.4 fold (p<0.0001), respectively. CPMV was unable to generate as strong an immune cell response compared to the H6/G3 conjugated CPMV nanoparticles in all tested immune cell categories; this was expected because CPMV did not accumulate in the lungs. The gating strategy used for the flow experiments can be found in FIG. 14.

### Discussion

Provided herein is a novel immunotherapeutic approach of utilizing cowpox mosaic virus (CPMV) as a cancer immunotherapy; specifically, in the context of an S100A9-targeted nanoparticle that homes to lungs in the pre-metastatic niche or after establishment of disease. The S100A9 nanoparticles demonstrate remarkable efficacy, not just as a treatment modality to reduce tumor burden when treating established disease, but most importantly, potent efficacy in the prophylactic setting is demonstrated. Applicant demonstrated efficacy of prophylaxis and therapy in lung metastasis models from melanoma and triple negative breast cancer. While the role of S100A9 in cancer has been recognized, targeting S100A9 with nanoparticles for cancer immunotherapy is a novel concept that could have broad implications. A central role of S100A9 in tumor aggressiveness has been established and S100A9 is expressed in lung metastasis as well as in many tumor types. Examples include but are not limited to including ovarian, skin, bladder, pancreatic, gastric, esophageal, colon, glioma, cervical, hepatocellular, and thyroid cancer. S100A9 is also expressed in a wide range of cell types. Examples include but are not limited to granulocytes, monocytes, osteoclasts, early myeloid lineage cells, platelets, and cancer cells. It can be expressed, secreted, or displayed, and secretion can be active or passive (i.e. neutrophil necrosis). These factors make S100A9 an attractive target and supports various tumor treatments to be targeted to S100A9. Further supporting this approach is the fact that S100A9 is secreted and found throughout the tumor microenvironment, making it an attractive target to direct nanoparticles and immunotherapies to tumors and metastatic disease. This disclosure relates to composition and treatments that recognize and exploit these new findings.

Applicant demonstrate herein that i.v. administered, S100A9-targeted CPMV homes to the lungs and that the CPMV nanoparticle adjuvant effectively immunomodulates the lung environment to recruit DCs and neutrophils while polarizing macrophages to the M1 phenotype protecting mice from i.v challenge with melanoma and TNBC. The S100A9-targeted CPMV also was effective to treat lung metastasis from melanoma or TNBC after establishment of the disease.

Targeted immunotherapies, such as the S100A9-targeted CPMV, can be a powerful treatment paradigm to treat high-risk patients and prevent metastatic outgrowth. The standard of care for metastatic cancer is chemotherapy, but this often fails due to development of resistance and/or necessary dose reduction due to harsh side effects.^{37,38} Alternatively, cancer immunotherapies have demonstrated that immune system modulation can result in dramatic antitumor activity. However, despite the enthusiasm surrounding clinical results using checkpoint inhibitor therapies,^{39,40} there continues to be a need to develop approaches that take advantage of neoantigens^{41,42} while overcoming therapy resistance.⁴³ Immunotherapies reversing the immunosuppressive TMEs can mitigate some of these challenges and can be used as solo or combination therapies to launch systemic anti-tumor immunity. It was demonstrated that CPMV as an in situ vaccine can act in this way, re-polarizing immunosuppressed environments and promoting immune cell recruitment and activation.^{28,44} Past studies have injected CPMV intratumorally against mouse models of ovarian, breast, colon cancer, and melanoma.⁴⁵⁻⁵⁰ The CPMV nanoparticles are recognized by innate immune cells and signal through pattern recognition receptors leading to release of immunostimulatory cytokines including interleukin (IL)-1β, IL-12, interferon (IFN)-γ chemokine ligand 3 (CCL3), macrophage inflammatory protein (MIP)-2, and granulocyte-macrophage colony-stimulating factor (GM-CSF) leading to monocyte recruitment.^{29,47} Activated DCs travel to nearby lymph nodes activating both CD4+ and CD8+ T-cells to establish immune memory. Therefore, direct intratumoral injection of CPMV is an effective strategy to induce systemic anti-tumor immunity, but is limited to injectable tumors. It is demonstrated herein that multivalent display of S100A9-targeting ligands directs the CPMV nanoparticles to the lung TME and induces treatment as evident by reduced tumor burden in the lungs after mice were i.v challenged using melanoma cells or TNBCs (FIG. 4).

S100A9 was used to target CPMV to the lungs because lung metastases are one of the most common sites of metastasis and prognosis is poor.⁵¹ In both men and women, the lungs were the third highest site of metastasis while in specific cancers such as genital cancers, metastatic growth to the lungs was the most common. Once metastasis occurs, survival rates are low and novel therapies to extend survival must be continuously researched and implemented.^{4,5} However, the concepts of targeting metastases in distant tissues could be expanded beyond just the lung. Many types of cancers including ovarian, skin, bladder, pancreatic, gastric, esophageal, colon, glioma, cervical, hepatocellular, and thyroid express S100A9.^{8,11-13} S100A9 is also expressed in a wide range of cell types including granulocytes, monocytes, osteoclasts, early myeloid lineage cells, platelets, and cancer cells.^{13,52} It can be expressed, secreted, or displayed, and secretion can be active or passive (i.e. neutrophil necrosis).^{17,52} The fact that S100A9 is secreted and found throughout the TME makes it an attractive target to direct nanoparticles and immunotherapies to the disease site (FIG. 3E). These design concepts could be applied to target other molecular signatures to tailor the nanoparticle treatment for organ-specific metastatic niches.

The CPMV platform technology is a versatile technology that could be adapted to target other molecular targets and/or deliver additional payloads.^{53,54} CPMV displaying peptide ligands specific for S100A9 were developed. Characterization of the S100A9-targeted nanoparticles of CPMV (as well as the CCMV control particles) demonstrated stable formulation chemistry, as PLC, and TEM which indicate the lack of substantial aggregation and structural uniformity of the viruses regardless of conjugation (FIG. 2 and FIG. 8). Denatured gels indicate a mosaic of conjugated and unconjugated coat proteins with up to 24 and 31 peptides per CPMV and CCMV nanoparticle, respectively. This equates to roughly 20 and 17% coat protein conjugation. Overall, the facile conjugation scheme producing monodisperse and highly conjugated viral nanoparticles is a key determinant in advancing the translatability and scalability of the CPMV platform.

The ability of the H6/G3 peptides to direct cargo to the TME was previously demonstrated when H6 and G3 peptides were conjugated to the Fc region of mouse IgG2b antibodies to specifically target S100A9 and deplete MDSCs within the TME.²² Previous work has also explored using small molecule drugs and neutralizing antibodies to block S100A9 function.^{8,21,22} However, to the best of Applicant's knowledge, S100A9 has never previously been targeted in immunotherapy. The exact functional role of S100A9 in cancer and tumorigenesis is not entirely understood, but the protein acts upon immune and tumor cells to modulate the TME into an immunosuppressive state thereby promoting tumor progression and aggressiveness.^{17,55-58} Without being bound by theory, targeting S100A9 could block its function halting tumor progression—however, S100A9-targeted CCMV particles showed no efficacy even after lung homing (29-35% distribution). Taking the biodistribution data into account, it is suggested that the therapeutic effect is achieved solely by the unique potency of CPMV with the S100A9 serving as solely as a molecular target.³² This was also validated by the RAW-BLUE™ data in that only the RAW-B™ cells were immunostimulated by CPMV and not CCMV and the peptides (FIG. 6A, FIG. 6B).

Especially encouraging was the fact that the S100A9-targeted CPMV treatment worked as both a prophylaxis and therapeutic immunotherapy (FIG. 4, FIG. 5). Prophylactically, the CPMV-H6 and CPMV-G3 formulations were able to decrease tumor nodules by 14.8 (p<0.0001) and 3.5-fold (p=0.0002) compared to PBS in the B16F10 murine melanoma model (FIG. 4C). Histological examination of the lungs also demonstrated that CPMV-H6 decreased the percentage of tumor cells by 18-fold (p<0.0001) while CPMV-G3 decreased it by 5-fold (p<0.0001). Similarly, when tested against a murine TNBC model, the CPMV-G3 particles decreased tumor nodule counts by 99-fold (p=0.0005) and delayed tumor growth (FIG. 4I, FIG. 12B). In both prophylactic studies, the CPMV particle without targeting showed some degrees of efficacy. In the melanoma study, CPMV decreased tumor nodules by 2.1-fold compared to PBS (p=0.02) while in the TNBC study, CPMV decreased tumor nodules by 10.6-fold (p=0.0015). This is most likely attributed to the ability of the CPMV nanoparticle to induce systemic immune responses;^{28,50} however, S100A9-targeted CPMV outperformed native CPMV. For instance, in the B16F10 repeat study, CPMV-H6 decreased tumor nodule counts by 3.3-fold (p=0.077) compared to native CPMV. The improved efficacy of the S100A9-targeted formulations can be attributed to tissue targeting resulting in modulation of the lung microenvironment where tumor metastasis occurs.

In the therapeutic studies, the targeted CPMV was similarly able to improve clinical outcomes in both the melanoma and breast cancer studies (FIG. 5). CPMV-H6 administration decreased B16F10 tumor nodules by 2.7-fold compared to PBS (p<0.0001) and 2.3-fold compared to native CPMV (FIG. 5B). In the 4T1-Luc study, CPMV-H6 slowed tumor growth and increased the median time of survival by 3 days (p=0.0077). Contrary to the prophylaxis studies, native CPMV showed insignificant benefit. Average B16F10 tumor nodule count with CPMV was 1.2-fold lower (p=0.36) compared to PBS, and CPMV was unable to slow 4T1-Luc tumor growth. This data indicates that systemic CPMV nanoparticles may have an ability to modulate immune-mediated clearance of circulating tumor cells; however, after tumor cells establish in tissue, localized immune-modulation is required.

The FACS data shed insight into the mechanisms behind the CPMV-induced immunogenicity (FIG. 6). Once the CPMV enters into the lung, it begins a cascade of events leading to stronger immune cell recruitment and activation (FIG. 6C). Specifically, DCs, neutrophils, and macrophages were recruited to the lungs by administration of CPMV-H6; DCs were increased by 40-50% (p<0.0001), neutrophils by 70-80% (p<0.0001), and macrophages by 50-70% (p<0.0001) compared to controls. Surprisingly, CPMV-G3 did not significantly improve immune cell recruitment; however, it did increase the number of active DCs by 12.1 fold (p<0.0001) and polarized 10.8 fold (p<0.0001) more M1 tumor-killing macrophages compared to PBS. M1 macrophages are potent tumor cell killers and have tumor-homing properties.⁵⁹ You et al. have shown that the number of M1 populations within the tumor islets in non-small cell lung cancer was positively correlated with patient survival.⁶⁰ DC activation can reduce immunosuppressive DC states and decrease tumorigenesis through the cross-priming of cytotoxic T-cells and the release of immunostimulatory cytokines such as IL-12, IFN-γ, and Fms-related tyrosine kinase 3 (FLT3).^{61,62}

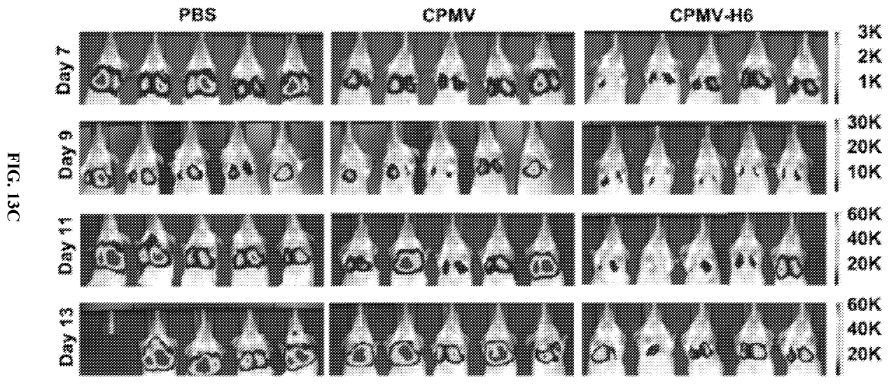

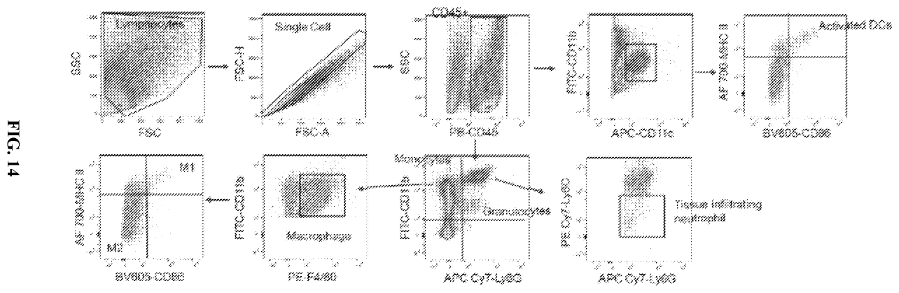

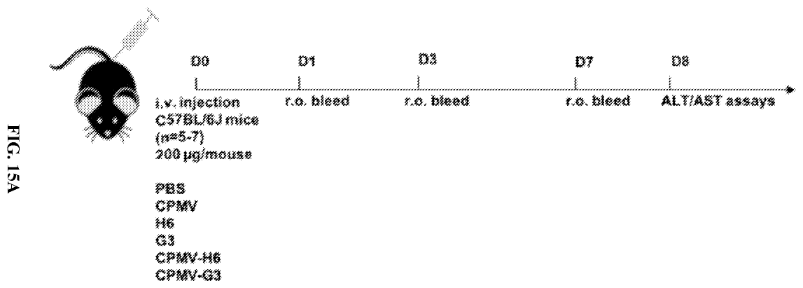

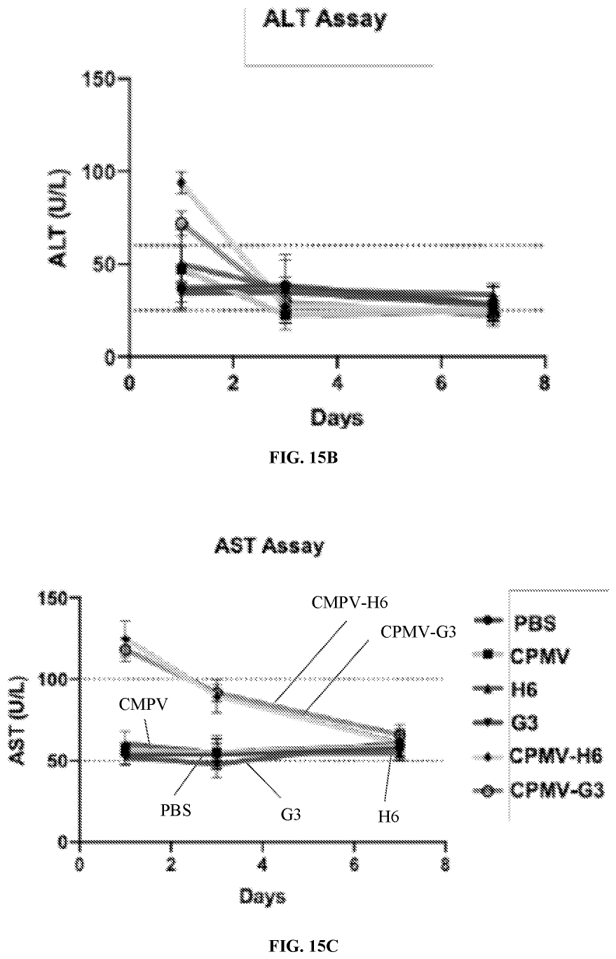

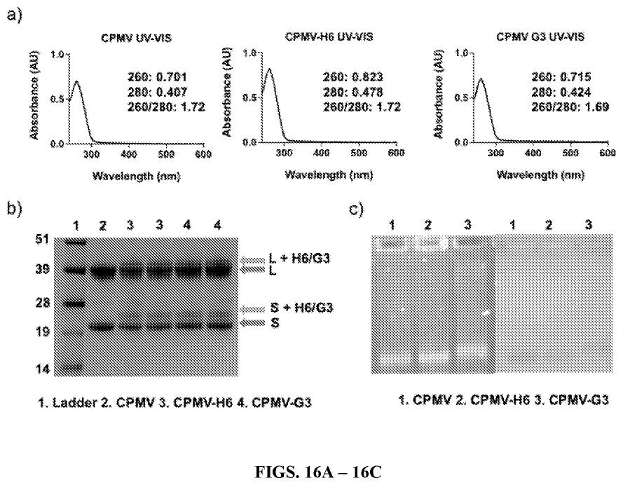

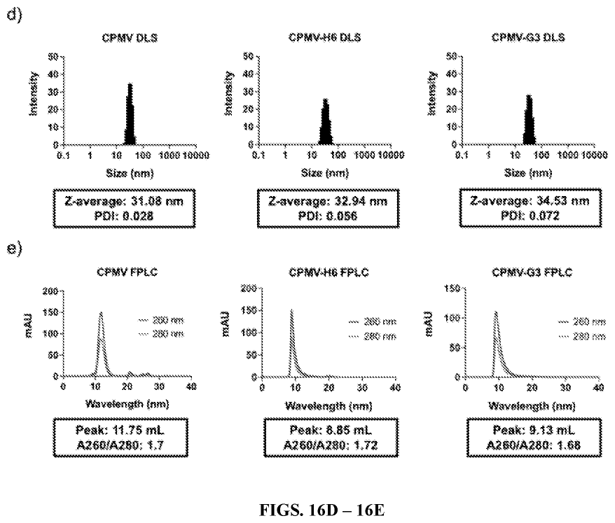

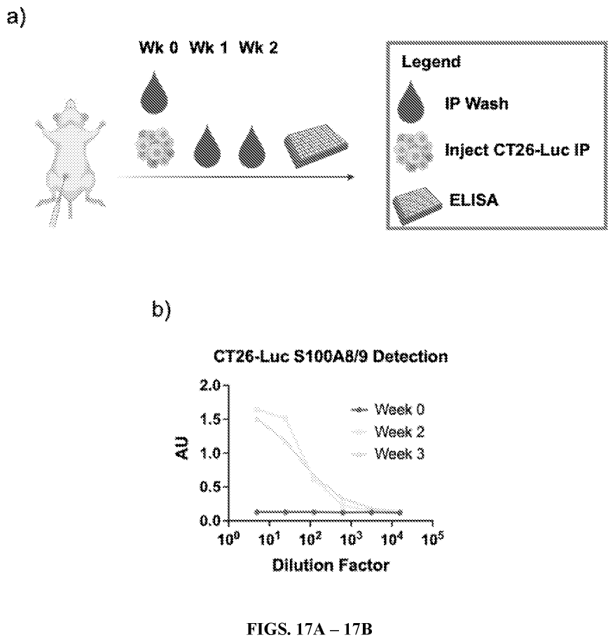

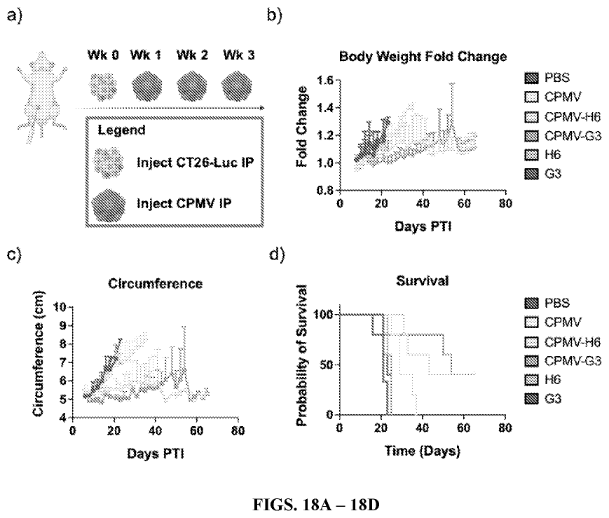

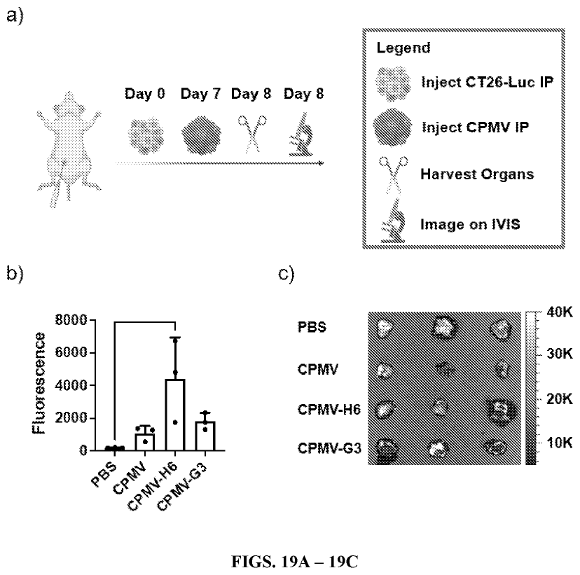

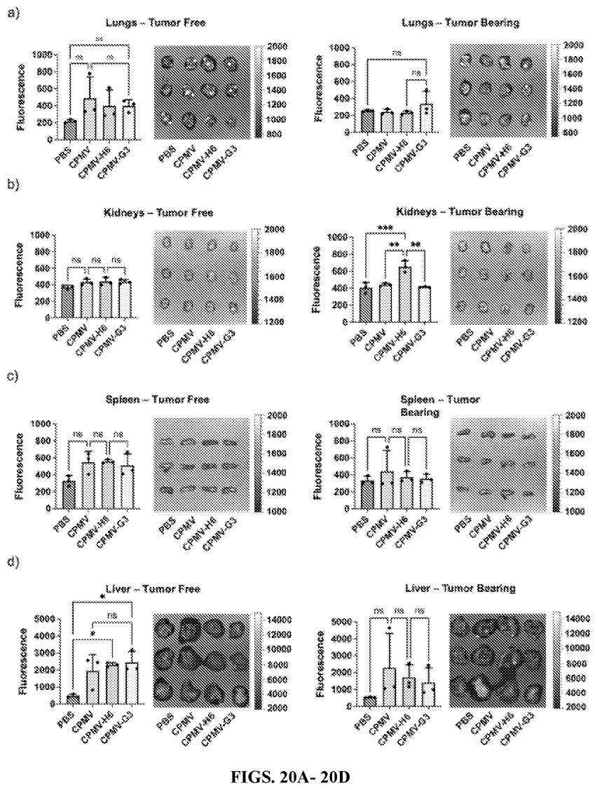

Together, data indicate that CPMV is a versatile cancer immunotherapy and its use could be extended beyond localized in situ treatments. As with other nanoparticle-based therapeutics i.v. administered CPMV is cleared by the liver, therefore Applicant also tested the hepatotoxicity by measuring serum alanine transaminase and aspartate transaminase (FIG. 15). After an initial increase in liver enzymes, physiological levels were restored within three days post treatment. In future studies more detailed immunotoxicity and pharmacology will be considered to pave the way for translational development. Further, combination therapies could be considered. Applicant has demonstrated that CPMV treatment synergizes with checkpoint blockade,⁶³ chemotherapy,⁴⁸ and radiation.^{25,27} Lastly, CPMV prime-boost administration schedules can be established or slow-release could be programmed through applications of long-lasting formulations with microneedles, polymers, scaffolds, or metal-organic frameworks.⁶⁴⁻⁶⁶

### Conclusion

Metastatic tumors remain one of the most challenging sectors in oncology to both treat and diagnose. S100A9 has been recognized as a targetable protein with high expression in multiple tumor types. Here it is demonstrate that S100A9-targeted nanoparticles from CPMV home to the lungs. When administered prior to tumor challenge, CPMV treatment acts as a prophylaxis and local immunomodulation (recruitment of DCs, neutrophils, and polarization of M1 macrophages) prevents lungs metastasis. The treatment was also effective when administered after disease establishment. This therapy can be a powerful prophylactic approach for high-risk patients, i.e. those undergoing surgery from primary melanoma or breast cancer, where recurrence and outgrowth of metastatic disease are the main clinical challenges.

### Materials and Cells

Potassium phosphate monobasic and dibasic salts, 20×3-morpholinepropane-1-sulfonic acid (MOPS) buffer, methanol, and acetic acid were purchased from Fisher Scientific. Dimethyl sulfoxide (DMSO) and sucrose were purchased from Sigma-Aldrich, 10× phosphate buffered saline (PBS) was purchased from G Biosciences, and sulfo-cyanine5-N-hydroxysuccinimide ester (sulfo-Cy5-NHS) was purchased from LumiProbe.

CT26-Luc cells were maintained in RPMI supplemented with 10% (v/v) fetal bovine serum (FBS) and 1% (v/v) penicillin/streptomycin. FBS and penicillin/streptomycin were purchased from Cytiva. The cells were maintained at 37° C. at 5% CO₂ levels.

### Production of S100A9-Targeted Cowpea Mosaic Virus Nanoparticles

Cowpea mosaic virus (CPMV) was isolated from infected black eyed pea plants as done previously^{[}70]. CPMV was stored in 100 mM potassium phosphate (KP) buffer pH 7.2 at 4° C. until later use. The H6 (MEWSLEKGYTIKGGGSC) and G3 (WGWSLSHGYQVKGGGSC) peptides were purchased from GenScript Biotech (San Diego), stored in DMSO, and conjugated to the CPMV virus nanoparticles (VNPs) as previously reported^{[}71]. In brief, an SMPEG₈ linker diluted in DMSO was added to CPMV at 5 equivalents per coat protein (CP) and incubated at room temperature (RT) for 2 h. The excess linker was removed through ultracentrifugation at 52 000 g for 1 h with a 40% (w/v) sucrose cushion. The pellet was resuspended in 10 mM KP (pH 7.2), and 1 equivalent per CP of the H6 or G3 peptide were added and reacted for 2 h at RT. Excess peptide was removed using a Sephadex G-25 MidiTrap column (Cytiva), and the resulting CPMV-H6 and CPMV-G3 were stored at 4° C. until further use.

### Characterization of S100A9-Targeted CPMV Nanoparticles

### Ultraviolet-Visible Spectroscopy (UV-VIS)

The concentrations of CPMV, CPMV-H6, and CPMV-G3 were determined using UV-VIS (NanoDrop). The absorbance values at 260 and 280 nm were measured, and the concentration of CPMV within the sample was calculated using the 260 nm wavelength and Beer's Law with an extinction coefficient of 8.1 mL mg⁻¹ cm⁻¹. The ratio of 260 to 280 nm (260 280⁻¹ of ˜1.8) was used to determine the absence of broken particles and protein contaminants within the samples.

### Sodium dodecyl sulfate-polyacrylamide Gel Electrophoresis (SDS-PAGE)

Both of the S100A9-targeted CPMV nanoparticles (NPs) and wild type (WT) CPMV were diluted in 10 mM KP and loaded with 4× lithium dodecyl sulfate Sample Buffer (Life Technologies) for a total concentration of 10 μg in 16 μL. The NPs were incubated at 95° C. for 5 min before loading onto a 12% NuPAGE gel (ThermoFisher Scientific). The gels were run at 200 V, 120 mA, and 25 W for 40 min in 1×MOPS buffer, and the protein stains were visualized using GelCode™ Blue Stain reagent (ThermoFisher Scientific) according to the manufacturer's instructions. The gels were then imaged under an AlphaImager System (Protein Simple).

### Agarose Gel Electrophoresis

The WT and S100A9-targeted CPMV NPs were diluted to a final concentration of 10 μg in 30 μL and run through a 1.2% (w/v) agarose gel stained with GelRed nucleic acid gel stain (Gold Biotechnology). The parameters for running the agarose gel were set at 120 V and 400 mA for 30 min. The RNA within the CPMV was then imaged using the AlphaImager System, and the gel was then stained overnight in 0.25% (w/v) Coomassie Blue followed by destaining in a solution of deionized water, methanol, and acetic acid in a 50:40:10 (v:v:v) ratio. The resulting protein bands were imaged using the AlphaImager System.

### Dynamic Light Scattering (DLS)

The size of the nanoparticles was measured using a Zetasizer Nano ZSP/Zen5600 (Malvern Panalytical) system. The samples were diluted to 0.1 mg mL⁻¹ in 10 mM KP and run at RT.

### Fast Protein Liquid Chromatography (FPLC)

FPLC (Akta Pure 25 M1, Cytiva) was used to determine the structural integrity and purity of the CPMV, CPMV-H6, and CPMV-G3 samples. The samples were diluted to a final concentration of 0.3 mg mL⁻¹ in 500 μL of 10 mM KP, and run through a Superose 6 size exclusion column with dimensions of 10×300 mm. The flow rate was set to 0.5 mg mL⁻¹ at an isocratic elution profile for a total elution volume of 50 mL, and absorbance measurements were taken at 260 and 280 nm to measure the nucleic acid and protein concentrations, respectively. The ratio of the absorbance values at 260 and 280 nm at the elution peak was calculated and compared to the UV-VIS values to ensure the structural integrity of the VNPs.

### Determination of S100A8/9 Expression Following Colon Cancer Injection

All mice experiments were carried out according to the guidelines set out by the Institutional Animal Care and Use Committee at the University of California, San Diego. The animals were purchased from Jackson Laboratory and stored at the Moores Cancer Center and provided with unlimited food and drink.

To ensure the presence and expression of S100A9 within the intraperitoneal (IP) space of mice following colon cancer injection, enzyme linked immunosorbent assays (ELISAs) were carried out on IP gavages of BALB/C mice. Female BALB/C mice (n=9) at 6-7 weeks old were purchased, and 3 of the 9 mice were immediately euthanized for IP gavage. The rest of the mice were injected IP with 500 000 CT26-Luc cells in 200 μL of PBS. Successful implantation of CT26-Luc tumors was verified by injecting 150 mg kg⁻¹ of D-luciferin (GoldBio Technologies) IP and imaging using luminescence with the in vivo imaging system (IVIS) by Xenogen. The IP fluid from the naïve mice was spun down at 10 000 g, and the supernatant was collected and stored at −80° C. until further use. Two weeks after tumor injection, 3 more mice were euthanized and the IP fluid was collected and stored as before. This was then repeated once more at the week 3 time point. The IP fluid was then analyzed using a mouse S100A8/9 DuoSet ELISA detection kit (R&D Systems) according to the manufacturer's instructions. An S100A8/9 kit was utilized as opposed to a monomeric S100A9 kit as S100A9 is mainly found within the body in its heterodimer form with S100A8^{[72]}.

### Colon Cancer Treatment Using S100A9-Targeted CPMV

The efficacy of S100A9-targeted CPMV was compared to WT CPMV and controls in an IP model of CT26-Luc. BALB/C mice at 6-7 weeks old were injected IP with 500 000 CT26-Luc cells in 200 μL of PBS. One week following tumor injection, mice were separated into WT CPMV, CPMV-H6, CPMV-G3, PBS, H6 peptide only, and G3 peptide only groups (n=5 per group), and injected with 200 μg of VNP diluted in 200 μL of PBS. The amount of H6 or G3 peptide injected was determined through densitometry analysis of the SDS-PAGE gels (ImageJ). The mice were re-injected at weeks 2 and 3 for a total of 3 injections. The circumference and body weight of the mice were measured every two days starting from the fourth day following tumor cell injection, and the survival of the mice was also followed. Mice were euthanized when their body weight fold change exceeded 75% of their original body weight or 30 g (whichever came first) or their circumference fold change exceeded 60% of their original circumference.

### Biodistribution of CPMV Particles Following IP Injection

To follow the biodistribution of the CPMV NPs within the reticuloendothelial organs, dual fluorescent and S100A9-targeted CPMV particles were produced. In these particles, equal molar excesses (5 equivalents per CP) of sulfo-Cy5-NHS and SMPEG₈ were added concomitantly, and excess Cy5 and SMPEG₈ were removed using ultracentrifugation as before. The H6 and G3 peptide were then added, and excess peptide was purified. For fluorescent CPMV without S100A9 targeting, 5 equivalents per CP of sulfo-Cy5-NHS was added without SMPEG₈. The particles were then analyzed using the same characterization methods as in Section 3 except fluorescent imaging was also undertaken during the agarose and SDS-PAGE characterization steps. For the UV-VIS and FPLC, absorbance measurements were also taken at 647 nm, and the amount of conjugated Cy5 per CPMV was calculated from UV-VIS using Beer's Law and the molar extinction coefficient of Cy5 (270 000 cm⁻¹ M⁻¹).

Female BALB/C mice were then injected IP with 500 000 CT26-Luc cells in 200 μL of PBS. After 1 week, 200 μg of the CPMV-Cy5, CPMV-Cy5-H6, and CPMV-Cy5-G3 particles diluted in 200 μL of PBS and a PBS control were injected IP (n=3), and the lungs, liver, kidneys, spleen, and tumors were harvested the following day. The organs were imaged using the IVIS, and fluorescent counts within the organs were calculated using region of interest (ROI) measurements within the Living Image 3.0 software. This experiment was also repeated in naïve mice without tumor burden to compare biodistribution across the organs in both tumor-bearing and naïve mice. Following imaging, all the organs were weighed and then homogenized with a hand-held homogenizer (Perkin Elmer) in 1 mL of PBS. The samples were spun down at 10 000 g for 10 min, and the supernatant was collected and read using a Tecan plate reader at 647 nm absorbance.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Lobbezoo, D. J. A. et al. Prognosis of metastatic breast cancer: are there differences between patients with de novo and recurrent metastatic breast cancer? Br J Cancer 112, 1445-1451 (2015).
2. Domingues, B., Lopes, J. M., Soares, P. & Pópulo, H. Melanoma treatment in review. Immunotargets Ther 7, 35-49 (2018).
3. Xiao, W. et al. Risk factors and survival outcomes in patients with breast cancer and lung metastasis: a population-based study. Cancer Med 7, 922-930 (2018).
4. Sandru, A., Voinea, S., Panaitescu, E. & Blidaru, A. Survival rates of patients with metastatic malignant melanoma. J Med Life 7, 572-576 (2014).
5. Chen, S. et al. Prognostic factors and survival outcomes according to tumor subtype in patients with breast cancer lung metastases. PeerJ 7, (2019).
6. Austin, E., Mamalis, A., Ho, D. & Jagdeo, J. Laser and light-based therapy for cutaneous and soft-tissue metastases of malignant melanoma: a systematic review. Arch. Dermatol. Res. 309, 229-242 (2017).
7. Widakowich, C., Castro, G. de, Azambuja, E. de, Dinh, P. & Awada, A. Review: Side Effects of Approved Molecular Targeted Therapies in Solid Cancers. The Oncologist 12, 1443-1455 (2007).
8. Gebhardt, C., Németh, J., Angel, P. & Hess, J. S100A8 and S100A9 in inflammation and cancer. Biochem. Pharmacol. 72, 1622-1631 (2006).
9. Wang, S. et al. S100A8/A9 in Inflammation. Front. Immunol. 9, (2018).
10. Gabrielsen, T. O. et al. Epidermal and dermal distribution of a myelomonocytic antigen (L1) shared by epithelial cells in various inflammatory skin diseases. J. Am. Acad. Dermatol. 15, 173-179 (1986).
11. Ryckman, C., Vandal, K., Rouleau, P., Talbot, M. & Tessier, P. A. Proinflammatory Activities of S100: Proteins S100A8, S100A9, and S100A8/A9 Induce Neutrophil Chemotaxis and Adhesion. The Journal of Immunology 170, 3233-3242 (2003).
12. Markowitz, J. & Carson, W. E. Review of S100A9 biology and its role in cancer. Biochim. Biophys. Acta 1835, 100-109 (2013).
13. Srikrishna, G. S100A8 and S100A9: new insights into their roles in malignancy. J Innate Immun 4, 31-40 (2012).
14. Edgeworth, J., Gorman, M., Bennett, R., Freemont, P. & Hogg, N. Identification of p8, 14 as a highly abundant heterodimeric calcium binding protein complex of myeloid cells. J. Biol. Chem. 266, 7706-7713 (1991).
15. Hunter, M. J. & Chazin, W. J. High level expression and dimer characterization of the S100 EF-hand proteins, migration inhibitory factor-related proteins 8 and 14. J. Biol. Chem. 273, 12427-12435 (1998).
16. Nacken, W., Sopalla, C., Pröpper, C., Sorg, C. & Kerkhoff, C. Biochemical characterization of the murine S100A9 (MRP14) protein suggests that it is functionally equivalent to its human counterpart despite its low degree of sequence homology. Eur. J. Biochem. 267, 560-565 (2000).
17. Németh, J. et al. S100A8 and S100A9 are novel nuclear factor kappa B target genes during malignant progression of murine and human liver carcinogenesis. Hepatology 50, 1251-1262 (2009).
18. Ichikawa, M., Williams, R., Wang, L., Vogl, T. & Srikrishna, G. S100A8/A9 activate key genes and pathways in colon tumor progression. Mol. Cancer Res. 9, 133-148 (2011).
19. Ehrchen, J. M., Sunderkötter, C., Foell, D., Vogl, T. & Roth, J. The endogenous Toll-like receptor 4 agonist S100A8/S100A9 (calprotectin) as innate amplifier of infection, autoimmunity, and cancer. J. Leukoc. Biol. 86, 557-566 (2009).
20. Hiratsuka, S. et al. The S100A8-serum amyloid A3-TLR4 paracrine cascade establishes a pre-metastatic phase. Nat. Cell Biol. 10, 1349-1355 (2008).
21. Bresnick, A. R. S100 proteins as therapeutic targets. Biophys Rev 10, 1617-1629 (2018).
22. Qin, H. et al. Generation of a new therapeutic peptide that depletes myeloid-derived suppressor cells in tumor-bearing mice. Nat. Med. 20, 676-681 (2014).
23. Murray, A. A., Wang, C., Fiering, S. & Steinmetz, N. F. In Situ Vaccination with Cowpea vs Tobacco Mosaic Virus against Melanoma. Mol Pharm 15, 3700-3716 (2018).
24. Shukla, S. et al. Plant viral nanoparticles-based HER2 vaccine: Immune response influenced by differential transport, localization and cellular interactions of particulate carriers. Biomaterials 121, 15-27 (2017).
25. Patel, R., Czapar, A. E., Fiering, S., Oleinick, N. L. & Steinmetz, N. F. Radiation Therapy Combined with Cowpea Mosaic Virus Nanoparticle in Situ Vaccination Initiates Immune-Mediated Tumor Regression. ACS Omega 3, 3702-3707 (2018).
26. Lizotte, P. H. et al. In situ vaccination with cowpea mosaic virus nanoparticles suppresses metastatic cancer. Nat Nanotechnol 11, 295-303 (2016).
27. Hoopes, P. J. et al. Treatment of Canine Oral Melanoma with Nanotechnology-Based Immunotherapy and Radiation. Mol. Pharmaceutics 15, 3717-3722 (2018).
28. Wang, C., Fiering, S. N. & Steinmetz, N. F. Cowpea Mosaic Virus Promotes Anti-Tumor Activity and Immune Memory in a Mouse Ovarian Tumor Model. Advanced Therapeutics 2, 1900003 (2019).
29. Lizotte, P. H. et al. In situ vaccination with cowpea mosaic virus nanoparticles suppresses metastatic cancer. Nat Nanotechnol 11, 295-303 (2016).
30. Cai, H., Wang, C., Shukla, S. & Steinmetz, N. F. Cowpea Mosaic Virus Immunotherapy Combined with Cyclophosphamide Reduces Breast Cancer Tumor Burden and Inhibits Lung Metastasis. Adv Sci (Weinh) 6, 1802281 (2019).
31. Wang, C., Fiering, S. N. & Steinmetz, N. F. Cowpea Mosaic Virus Promotes Anti-Tumor Activity and Immune Memory in a Mouse Ovarian Tumor Model. Advanced Therapeutics 2, 1900003 (2019).
32. Shukla, S. et al. The unique potency of Cowpea mosaic virus (CPMV) in situ cancer vaccine. Biomater. Sci. 8, 5489-5503 (2020).
33. Gillitzer, E., Willits, D., Young, M. & Douglas, T. Chemical modification of a viral cage for multivalent presentation. Chemical Communications 0, 2390-2391 (2002).
34. Chatterji, A. et al. New addresses on an addressable virus nanoblock; uniquely reactive Lys residues on cowpea mosaic virus. Chem Biol 11, 855-863 (2004).
35. Lam, P. & Steinmetz, N. F. Delivery of siRNA therapeutics using cowpea chlorotic mottle virus-like particles. Biomater. Sci. 7, 3138-3142 (2019).
36. Singh, P. et al. Bio-distribution, toxicity and pathology of cowpea mosaic virus nanoparticles in vivo. J Control Release 120, 41-50 (2007).
37. Yoneda, K. Y., Louie, S. & Shelton, D. K. Approach to pulmonary metastases. Curr Opin Pulm Med 6, 356-363 (2000).
38. Ashdown, M. L. et al. Chemotherapy for Late-Stage Cancer Patients: Meta-Analysis of Complete Response Rates. F1000Res 4, (2015).
39. Szostak, B., Machaj, F., Rosik, J. & Pawlik, A. CTLA4 antagonists in phase I and phase II clinical trials, current status and future perspectives for cancer therapy. Expert Opin Investig Drugs 28, 149-159 (2019).
40. Johnson, C. B. & Win, S. Y. Combination therapy with PD-1/PD-L1 blockade: An overview of ongoing clinical trials. Oncoimmunology 7, e1408744 (2018).
41. Schumacher, T. N. & Schreiber, R. D. Neoantigens in cancer immunotherapy. Science 348, 69-74 (2015).
42. Castle, J. C., Uduman, M., Pabla, S., Stein, R. B. & Buell, J. S. Mutation-Derived Neoantigens for Cancer Immunotherapy. Front Immunol 10, 1856 (2019).
43. Fares, C. M., Van Allen, E. M., Drake, C. G., Allison, J. P. & Hu-Lieskovan, S. Mechanisms of Resistance to Immune Checkpoint Blockade: Why Does Checkpoint Inhibitor Immunotherapy Not Work for All Patients? Am Soc Clin Oncol Educ Book 39, 147-164 (2019).
44. Wang, C., Beiss, V. & Steinmetz, N. F. Cowpea Mosaic Virus Nanoparticles and Empty Virus-Like Particles Show Distinct but Overlapping Immunostimulatory Properties. Journal of Virology 93, (2019).
45. Kerstetter-Fogle, A. et al. Plant Virus-Like Particle In Situ Vaccine for Intracranial Glioma Immunotherapy. Cancers (Basel) 11, (2019).
46. Shukla, S., Wang, C., Beiss, V. & Steinmetz, N. F. Antibody Response against Cowpea Mosaic Viral Nanoparticles Improves In Situ Vaccine Efficacy in Ovarian Cancer. ACS Nano 14, 2994-3003 (2020).
47. Murray, A. A., Wang, C., Fiering, S. & Steinmetz, N. F. In Situ Vaccination with Cowpea vs Tobacco Mosaic Virus against Melanoma. Mol Pharm 15, 3700-3716 (2018).
48. Cai, H., Wang, C., Shukla, S. & Steinmetz, N. F. Cowpea Mosaic Virus Immunotherapy Combined with Cyclophosphamide Reduces Breast Cancer Tumor Burden and Inhibits Lung Metastasis. Adv Sci (Weinh) 6, 1802281 (2019).
49. Cai, H., Shukla, S., Wang, C., Masarapu, H. & Steinmetz, N. F. Heterologous Prime-Boost Enhances the Antitumor Immune Response Elicited by Plant-Virus-Based Cancer Vaccine. J Am Chem Soc 141, 6509-6518 (2019).
50. Lizotte, P. H. et al. In situ vaccination with cowpea mosaic virus nanoparticles suppresses metastatic cancer. Nat Nanotechnol 11, 295-303 (2016).
51. Riihimäki, M., Thomsen, H., Sundquist, K., Sundquist, J. & Hemminki, K. Clinical landscape of cancer metastases. Cancer Med 7, 5534-5542 (2018).
52. Ghoneum, A., Afify, H., Salih, Z., Kelly, M. & Said, N. Role of tumor microenvironment in the pathobiology of ovarian cancer: Insights and therapeutic opportunities. Cancer Med 7, 5047-5056 (2018).
53. Aljabali, A. A. A., Shukla, S., Lomonossoff, G. P., Steinmetz, N. F. & Evans, D. J. CPMVDOX delivers. Mol. Pharm. 10, 3-10 (2013).
54. Miermont, A. et al. Cowpea Mosaic Virus Capsid, a Promising Carrier for the Development of Carbohydrate Based Anti-tumor Vaccines. Chemistry 14, 4939-4947 (2008).
55. Källberg, E. et al. S100A9 Interaction with TLR4 Promotes Tumor Growth. PLOS ONE 7, e34207 (2012).
56. Ghavami, S. et al. S100A8/A9 at low concentration promotes tumor cell growth via RAGE ligation and MAP kinase-dependent pathway. J. Leukoc. Biol. 83, 1484-1492 (2008).
57. Ridnour, L. A. et al. Molecular pathways: toll-like receptors in the tumor microenvironment-poor prognosis or new therapeutic opportunity. Clin. Cancer Res. 19, 1340-1346 (2013).
58. Hermani, A. et al. Calcium-binding proteins S100A8 and S100A9 as novel diagnostic markers in human prostate cancer. Clin. Cancer Res. 11, 5146-5152 (2005).
59. Parayath, N. N., Parikh, A. & Amiji, M. M. Repolarization of Tumor-Associated Macrophages in a Genetically Engineered Nonsmall Cell Lung Cancer Model by Intraperitoneal Administration of Hyaluronic Acid-Based Nanoparticles Encapsulating MicroRNA-125b. Nano Lett. 18, 3571-3579 (2018).
60. Ma, J. et al. The M1 form of tumor-associated macrophages in non-small cell lung cancer is positively associated with survival time. BMC Cancer 10, 112 (2010).
61. Hochrein, H. et al. Differential production of IL-12, IFN-alpha, and IFN-gamma by mouse dendritic cell subsets. J. Immunol. 166, 5448-5455 (2001).
62. Karsunky, H., Merad, M., Cozzio, A., Weissman, I. L. & Manz, M. G. Flt3 Ligand Regulates Dendritic Cell Development from Flt3+Lymphoid and Myeloid-committed Progenitors to Flt3+ Dendritic Cells In Vivo. J Exp Med 198, 305-313 (2003).
63. Wang, C. & Steinmetz, N. F. A Combination of Cowpea Mosaic Virus and Immune Checkpoint Therapy Synergistically Improves Therapeutic Efficacy in Three Tumor Models. Advanced Functional Materials 30, 2002299 (2020).
64. Cai, H., Shukla, S., Wang, C., Masarapu, H. & Steinmetz, N. F. Heterologous Prime-Boost Enhances the Antitumor Immune Response Elicited by Plant-Virus-Based Cancer Vaccine. J. Am. Chem. Soc. 141, 6509-6518 (2019).
65. Czapar, A. E., Tiu, B. D. B., Veliz, F. A., Pokorski, J. K. & Steinmetz, N. F. Slow-Release Formulation of Cowpea Mosaic Virus for In Situ Vaccine Delivery to Treat Ovarian Cancer. Advanced Science 5, 1700991 (2018).
66. Luzuriaga, M. A. et al. Enhanced Stability and Controlled Delivery of MOF-Encapsulated Vaccines and Their Immunogenic Response In Vivo. ACS Applied Materials & Interfaces (2019) doi: 10.1021/acsami.8b20504.
67. Wen, A. M. et al. Viral Nanoparticles for In vivo Tumor Imaging. J Vis Exp (2012) doi: 10.3791/4352.
68. Bankhead, P. et al. QuPath: Open source software for digital pathology image analysis Scientific Reports 7, 16878 (2017).
69. Huang, X.-J. et al. Aspartate Aminotransferase (AST/GOT) and Alanine Aminotransferase (ALT/GPT) Detection Techniques. Sensors (Basel) 6, 756-782 (2006).
70. Wen, A. M., et al., Viral Nanoparticles for In vivo Tumor Imaging, J. Vis. Exp., (69), e4352, (2012).
71. Chung, Y. H., et al., S100A9-Targeted Cowpea Mosaic Virus as a Prophylactic and Therapeutic Immunotherapy against Metastatic Breast Cancer and Melanoma, Adv. Sci., (8), 21, e2101796, (2021).
72. Wang, S., et al., S100A8/A9 in Inflammation, Front. Immnol., (9), 1298, (2018).

1. A nanoparticle comprising a virus or virus like particle (VLP) and a peptide that targets or recognizes and binds S100A9.

2. The nanoparticle of claim 1, wherein the virus or VLP is from a plant virus from the group of the genus Bromovirus, Comovirus, or Tymovirus.

3. The nanoparticle of claim 2, wherein the plant virus is selected from Cowpea chlorotic mottle virus (CCMV), Cowpea mosaic virus (CPMV), or Physalis mottle virus (PhMV).

4. The nanoparticle of claim 1, wherein the virus or VLP has an exposed lysine side chain.

5. The nanoparticle of claim 1, wherein the peptide that targets or recognizes and binds S100A9 comprises a linker or a c-terminal cysteine and optionally wherein a N-hydroxysuccinimide (NHS) ester conjugates with the lysine side chain and a maleimide of a maleimide-polyethylene glycol₈ (SM(PEG)₈) conjugates with the c-terminal cysteine of the peptide.

6-7. (canceled)

8. The nanoparticle of claim 1, wherein the peptide that targets or recognizes and binds S100A9 comprises any one or more of SEQ ID NO: 1 and/or 2 or 1, 2 or 10 to 15, or a peptide having at least 70% sequence identity thereto.

9. A polynucleotide encoding the nanoparticle of claim 1.

10. A vector or host cell comprising the polynucleotide of claim 9.

11. (canceled)

12. A plurality of the nanoparticles of claim 1, wherein the nanoparticles are the same or different from each other.

13. A composition comprising the nanoparticle of claim 1, and a carrier and optionally an additional therapeutic agent.

14. (canceled)

15. A method for inducing an immune response in a subject in need thereof comprising administering to the subject: the nanoparticle of claim 1.

16. A method for targeting the tumor microenvironment to reverse immunosuppression in a tumor that optionally expresses or secretes S100A, comprising contacting the tumor with the nanoparticle of claim 1.

17. (canceled)

18. A method for treating cancer in a subject in need thereof, comprising administering to the subject: the nanoparticle of claim 1 and optionally wherein the cancer is selected from a primary or metastatic melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer.

19-21. (canceled)

22. The method of claim 18, wherein the cancer is metastatic melanoma or metastatic triple negative breast cancer and optionally wherein the cancer is metastatic cancer that is present in the lung of the subject.

23-24. (canceled)

25. The method of claim 22, wherein the cancer expresses S100A9.

26. The method of claim 25, further comprising the administration of a different cancer therapy or tumor resection.

27-28. (canceled)

29. A method of altering an immune cell profile in lungs or intraperitoneal space of a subject comprising administering to the subject an effective amount of the nanoparticle of any of the nanoparticle of claim 1.

30. (canceled)

31. A kit comprising one or more of the nanoparticle of any one of claim 1 and optional instructions for use.
